(19) United States
(12) Patent Application Publication
(10) Pub. No.: US 2022/0010322 A1
(43) Pub. Date: Jan. 13, 2022

(54) GENE SILENCING VIA GENOME EDITING

(71) Applicant: SYNGENTA CROP PROTECTION AG, Basel (CH)

(72) Inventors: Jian LV, Beijing (CN); Xi CHEN, Beijing (CN); Kun YU, Beijing (CN); Dawei LIANG, Beijing (CN); Hongju ZHOU, Beijing (CN); Jianping XU, Beijing (CN)

(73) Assignee: SYNGENTA CROP PROTECTION AG, Basel (CH)

(21) Appl. No.: 17/297,527

(22) PCT Filed: Nov. 26, 2019
(86) PCT No.: PCT/US2019/063200

§ 371 Date: May 27, 2021

(30) Foreign Application Priority Data
Dec. 4, 2018 (CN) PCT/CN2018/119155

(51) Int. Cl. C12N 15/82

(52) U.S. Cl. CPC C12N 15/8213; C12N 2800/80

(57) ABSTRACT

The present invention relates to methods and compositions for gene silencing by genome editing. In some embodiments, nucleases are provided selected from the group consisting of meganucleases (MNs), zinc-finger nucleases (ZFNs), transcription-activator like effector nucleases (TALENs), Cas9 nuclease, Cfp1 nuclease, dCas9-FokI, dCpf1-FokI, chimeric Cas9/Cpf1-cytidine deaminase, chimeric Cas9/Cpf1-adenine deaminase, chimeric FEN1-FokI, and Mega-TALs, a nickase Cas9 (nCas9), chimeric dCas9 non-FokI nuclease and dCpf1 non-FokI nuclease. Additionally, the present invention relates to methods and compositions for gene silencing by genome editing. Also provided are methods and compositions for rearranging a chromosome by genome editing.

### RELATED APPLICATIONS

This application claims the benefit of PCT/CN2018/119155 filed Dec. 4, 2018 and incorporated by reference in its entirety herein.

### SEQUENCE LISTING

A Sequence Listing in ASCII text format, submitted under 37 C.F.R. § 1.821, entitled “81724_ST25.txt”, 47 kilobytes in size, generated on Nov. 19, 2018. This Sequence Listing is hereby incorporated by reference into the specification for its disclosures.

### FIELD OF THE INVENTION

The present invention relates to methods and compositions for gene silencing by genome editing or rearranging a chromosome by genome editing.

### BACKGROUND OF THE INVENTION

Gene silencing is a key tool to study gene function and deliver key traits in crops. Traditional strategies for gene silencing in plants include (Cold Spring Harb Symp Quant Biol. 2006; 71:481-5), transgenic over-expression of sense RNA transcript, transgenic expression of a hairpin transcript, transgenic expression of an antisense RNA transcript. Meanwhile, there are some limitations for the technologies. For overproduced sense transcripts, the transcript should be without premature stop codon, otherwise the efficacy is not good enough and not stable. While for hairpin and antisense design, matching expression of silencing RNA at same tissue and at same develop stage as natural target mRNA is impossible; so the RNA silencing effect is leaky.

This disclosure provides a novel gene silencing method using genome editing to create chromosome inversions. Genome editing has not been used in gene silencing except to bring transcriptional regulators close to the promoter of a gene to be silenced as in WO18057863, WO2017180915, and WO2017023974

### SUMMARY

The present disclosure provides a method of reducing expression a target gene comprised of, introducing into a cell a nuclease capable of site-directed DNA cleavage at a target genomic site, making two or more double strand cuts within a single target gene, selecting for a cell where the double strand cuts have been repaired with the intervening DNA inverted, and reducing expression of the target gene. In some embodiments, the nuclease is selected from the group consisting of meganucleases (MNs), zinc-finger nucleases (ZFNs), transcription-activator like effector nucleases (TALENs), Cas9 nuclease, Cfp1 nuclease, dCas9-FokI, dCpf1-FokI, chimeric Cas9/Cpf1-cytidine deaminase, chimeric Cas9/Cpf1-adenine deaminase, chimeric FEN1-FokI, and Mega-TALs, a nickase Cas9 (nCas9), chimeric dCas9 non-FokI nuclease and dCpf1 non-FokI nuclease. In some embodiments of the method, the double strand cuts in the target gene are located in the promoter, UTR, exon, intron, or gene-gene junction region. These methods may be used when the cell has a haploid, diploid, polyploid, or hexiploid genome. These methods may be used when the target gene is dominant, recessive, or semi-dominant. In some embodiments the method may make use of one, two or more guide sequences. This method is useful in plant cells, but is applicable to any cell.

The present disclosure provides a method of rearranging a chromosome by genome editing comprising generating at least one breakage in the chromosome by a site-directed nuclease, selecting a chromosome with a rearrangement. In some embodiments, the method can utilize a site-directed nuclease is selected from the group consisting of meganucleases (MNs), zinc-finger nucleases (ZFNs), transcription-activator like effector nucleases (TALENs), Cas9 nuclease, Cfp1 nuclease, dCas9-FokI, dCpf1-FokI, chimeric Cas9/Cpf1-cytidine deaminase, chimeric Cas9/Cpf1-adenine deaminase, chimeric FEN1-FokI, and Mega-TALs, a nickase Cas9 (nCas9), chimeric dCas9 non-FokI nuclease and dCpf1 non-FokI nuclease. In some embodiment of the method, the chromosome rearrangement comprises a deletion, duplication, inversion, or translocation. In some embodiments of the method, the chromosome rearrangement causes a modification of gene expression. In some embodiments of the method, the gene expression modification includes regulation at precursor mRNA level, or at mature mRNA level or at translation level. In some embodiments of the method, the chromosome rearrangement includes chromosomes from two species when the chromosomes can be grouped in one nuclei such as in an interspecific hybrid. In some embodiments of the method, the chromosome rearrangement leads to new allele generation via fusing at least two alleles or two components from different alleles. In some embodiments of the method, chromosome rearrangement is targeted to a promoter, exon, intron, or transcription terminator. In some embodiments of the method, chromosome rearrangement causes a modification of gene expression of different genes with sequence similarity to the rearranged gene. In further embodiments of the method, the deletion, duplication, inversion, or translocation is no less than 19 base pairs.

### BRIEF DESCRIPTION OF THE DRAWINGS

FIG. 1 graphical representation of transformation events with construct 22602 (left) where two edits in Exon5 resulted in deletion in event RIET142202A130A or inversion in event RIET142202A049A and representation of transformation events with construct 22604 (right) where two edits in Exon5 resulted in deletion in event RIET142300A014A or inversion in event RIET142500B024A049A.

FIG. 2 shows a workflow to measure expression of DEP1 from the endogenous locus.

FIG. 3 Gel electrophoresis of the PCR products from genotyping of T1 seeds from event RIET142202A130A.

FIG. 4 Wild type rice plant next to plant with deletion in DEP1.

FIG. 5 2-3 cm rice plant sampled for RNA isolation.

FIG. 6 Gel electrophoresis of rtPCR products from FI plants 17SBC500140, 17SBC500143, 17SBC500146, and 17SBC500149.

FIG. 7 Graphical representations of exon 5 of DEP1 in F1 plants with deletions or insertions (above) and rtPCR products separated by gel electrophoresis (below) the DEP1 products quantified and normalized to the ratio of expression of the rice ubiquitin gene OS03g0234200.

FIG. 8 Graphical representations of exon 5 of DEP1 from E0 plant, RIET142500A084A, identified with two translocations.

FIG. 9 Schematic showing how two or more genome editing targets can be used to select for chromosomal translocations and duplications.

FIG. 10 Schematic showing how inversions can lead to gene silencing.

FIG. 11 Schematic showing how silencing by chromosomal inversion can be used to silence gene orthologs in hexaploid or polyploid genomes.

### BRIEF DESCRIPTION OF THE SEQUENCES IN THE SEQUENCE LISTING

SEQ ID NO. 1 is the coding sequence of DENSE AND ERECT PANICLE 1

SEQ ID NO. 2 is the gRNA-B and gRNA-D expression cassette from vector 22603

SEQ ID NO. 3 is the guide RNA-B targeting exon 5 of DEP1

SEQ ID NO. 4 is the gRNA-D also targeting exon 5

SEQ ID NO. 5 is the 22604 gRNA-A, gRNA-D, gRNA-B, and gRNA-C

SEQ ID NO. 6 is the guide RNA-A

SEQ ID NO. 7 is the guide RNA-C

SEQ ID NO. 8 is the CAS9 expression cassette

SEQ ID NO. 9 is the CAS9 Taqman Assay Forward Primer

SEQ ID NO. 10 is the CAS9 Taqman Assay Reverse Primer

SEQ ID NO. 11 is the CAS9 Taqman Assay Probe

SEQ ID NO. 12 is the gRNA-A Taqman Assay Forward Primer

SEQ ID NO. 13 is the gRNA-A Taqman Assay Reverse Primer

SEQ ID NO. 14 is the gRNA-A Taqman Assay Probe

SEQ ID NO. 15 is the gRNA-C Taqman Assay Forward Primer

SEQ ID NO. 16 is the gRNA-C Taqman Assay Reverse Primer

SEQ ID NO. 17 is the gRNA-C Taqman Assay Probe

SEQ ID NO. 18 is the gRNA-D Taqman Assay Forward Primer

SEQ ID NO. 19 is the gRNA-D Taqman Assay Reverse Primer

SEQ ID NO. 20 is the gRNA-D Taqman Assay Probe

SEQ ID NO. 21 is the DEP1 PCR primer 1

SEQ ID NO. 22 is the DEP1 PCR primer 2

SEQ ID NO. 23 is the inversion between RNA-A and RNA-B

SEQ ID NO. 24 is the deletion between gRNA-B and gRNA-D

SEQ ID NO. 25 is the an inversion between gRNA-A and gRNA-B

SEQ ID NO. 26 is the RIET142300A014A was selected as expression control

SEQ ID NO. 27 is the sense genotyping primers for 14SBC500773

SEQ ID NO. 28 is the anti-sense genotyping primers for 14SBC500773

SEQ ID NO. 29 is the DEP1 qRT-PCR sense primer, locating in exon 1

SEQ ID NO. 30 is the DEP1 qRT-PCR antisense primer, locating in 3′ UTR

SEQ ID NO. 31 is the Rice ubiquitin (OS03g0234200) qRT-PCR primer 1

SEQ ID NO. 32 is the Rice ubiquitin (OS03g0234200) qRT-PCR primer 2

SEQ ID NO. 33 is the wild-type DEPT qRT-PCR primer 1

SEQ ID NO. 34 is the wild-type DEP1 qRT-PCR primer 2

SEQ ID NO. 35 is the translocation between gRNA-A and gRNA-B, gRNA-C and gRNA-D

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

The following definitions and methods are provided to better define the present invention and to guide those of ordinary skill in the art in the practice of the present invention. Unless otherwise noted, terms used herein are to be understood according to conventional usage by those of ordinary skill in the relevant art. Definitions of common terms in molecular biology may also be found in Rieger et al., Glossary of Genetics: Classical and Molecular, 5^{th} edition, Springer-Verlag, New York, 1994.

As used in the description of the embodiments of the invention and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount.

The terms “comprise,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the term “amplified” means the construction of multiple copies of a nucleic acid molecule or multiple copies complementary to the nucleic acid molecule using at least one of the nucleic acid molecules as a template. See, e.g., Diagnostic Molecular Microbiology: Principles and Applications, D. H. Persing et al., Ed., American Society for Microbiology, Washington, D.C. (1993). The product of amplification is termed an amplicon.

A “coding sequence” is a nucleic acid sequence that is transcribed into RNA such as mRNA, rRNA, tRNA, snRNA, sense RNA or antisense RNA. In some embodiments, the RNA is then translated in an organism to produce a protein.

As used herein the term transgenic “event” refers to a recombinant plant produced by transformation and regeneration of a single plant cell with heterologous DNA, for example, an expression cassette that includes one or more genes of interest (e.g., transgenes). The term “event” refers to the original transformant and/or progeny of the transformant that include the heterologous DNA. The term “event” also refers to progeny produced by a sexual outcross between the transformant and another line. Even after repeated backcrossing to a recurrent parent, the inserted DNA and the flanking DNA from the transformed parent is present in the progeny of the cross at the same chromosomal location. Normally, transformation of plant tissue produces multiple events, each of which represent insertion of a DNA construct into a different location in the genome of a plant cell. Based on the expression of the transgene or other desirable characteristics, a particular event is selected. Thus, “event MIR604,” “MIR604” or “MIR604 event” as used herein, means the original MIR604 transformant and/or progeny of the MIR604 transformant (U.S. Pat. Nos. 7,361,813, 7,897,748, 8,354,519, and 8,884,102, incorporated by references herein).

“Expression cassette” as used herein means a nucleic acid molecule capable of directing expression of a particular nucleotide sequence in an appropriate host cell, comprising a promoter operably linked to the nucleotide sequence of interest, typically a coding region, which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette may also comprise sequences not necessary in the direct expression of a nucleotide sequence of interest but which are present due to convenient restriction sites for removal of the cassette from an expression vector. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation process known in the art. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development. An expression cassette, or fragment thereof, can also be referred to as “inserted sequence” or “insertion sequence” when transformed into a plant.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. Genes can include both coding and non-coding regions (e.g., introns, regulatory elements, promoters, enhancers, termination sequences and 5′ and 3′ untranslated regions). A gene typically expresses mRNA, functional RNA, or specific protein, including regulatory sequences. Genes may or may not be capable of being used to produce a functional protein. In some embodiments, a gene refers to only the coding region. The term “native gene” refers to a gene as found in nature. The term “chimeric gene” refers to any gene that contains 1) DNA sequences, including regulatory and coding sequences that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

By the term “express” or “expression” of a polynucleotide coding sequence, it is meant that the sequence is transcribed, and optionally translated.

A “gene of interest”, “nucleotide sequence of interest”, or “sequence of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

As used herein, “heterologous” refers to a nucleic acid molecule or nucleotide sequence not naturally associated with a host cell into which it is introduced, that either originates from another species or is from the same species or organism but is modified from either its original form or the form primarily expressed in the cell, including non-naturally occurring multiple copies of a naturally occurring nucleic acid sequence. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., present in a different copy number, and/or under the control of different regulatory sequences than that found in the native state of the nucleic acid molecule. A nucleic acid sequence can also be heterologous to other nucleic acid sequences with which it may be associated, for example in a nucleic acid construct, such as e.g., an expression vector. As one non-limiting example, a promoter may be present in a nucleic acid construct in combination with one or more regulatory element and/or coding sequences that do not naturally occur in association with that particular promoter, i.e., they are heterologous to the promoter.

A “homologous” nucleic acid sequence is a nucleic acid sequence naturally associated with a host cell into which it is introduced. A homologous nucleic acid sequence can also be a nucleic acid sequence that is naturally associated with other nucleic acid sequences that may be present, e.g., in a nucleic acid construct. As one non-limiting example, a promoter may be present in a nucleic acid construct in combination with one or more regulatory elements and/or coding sequences that naturally occur in association with that particular promoter, i.e. they are homologous to the promoter.

“Operably-linked” refers to the association of nucleic acid sequences on a single nucleic acid sequence so that the function of one affects the function of the other. For example, a promoter is operably-linked with a coding sequence or functional RNA when it is capable of affecting the expression of that coding sequence or functional RNA (i.e. the coding sequence or functional RNA is under the transcriptional control of the promoter). Coding sequences in sense or antisense orientation can be operably-linked to regulatory sequences. Thus, regulatory or control sequences (e.g., promoters) operatively associated with a nucleotide sequence are capable of effecting expression of the nucleotide sequence. For example, a promoter operably linked to a nucleotide sequence encoding GFP would be capable of effecting the expression of that GFP nucleotide sequence.

The control sequences need not be contiguous with the nucleotide sequence of interest, as long as they function to direct the expression thereof. Thus, for example, intervening untranslated, yet transcribed, sequences can be present between a promoter and a coding sequence, and the promoter sequence can still be considered “operably linked” to the coding sequence.

“Primers” as used herein are isolated nucleic acids that are annealed to a complementary target DNA strand by nucleic acid hybridization to form a hybrid between the primer and the target DNA strand, then extended along the target DNA strand by a polymerase, such as DNA polymerase. Primer pairs or sets can be used for amplification of a nucleic acid molecule, for example, by the polymerase chain reaction (PCR) or other nucleic-acid amplification methods.

A “probe” is an isolated nucleic acid molecule that is complementary to a portion of a target nucleic acid molecule and is typically used to detect and/or quantify the target nucleic acid molecule. Thus, in some embodiments, a probe can be an isolated nucleic acid molecule to which is attached a detectable moiety or reporter molecule, such as a radioactive isotope, ligand, chemiluminescence agent, fluorescence agent or enzyme. Probes according to the present invention can include not only deoxyribonucleic or ribonucleic acids but also polyamides and other probe materials that bind specifically to a target nucleic acid sequence and can be used to detect the presence of and/or quantify the amount of, that target nucleic acid sequence.

A TaqMan probe is designed such that it anneals within a DNA region amplified by a specific set of primers. As the Taq polymerase extends the primer and synthesizes the nascent strand from a single-strand template from 3′ to 5′ of the complementary strand, the 5′ to 3′ exonuclease of the polymerase extends the nascent strand through the probe and consequently degrades the probe that has annealed to the template. Degradation of the probe releases the fluorophore from it and breaks the close proximity to the quencher, thus relieving the quenching effect and allowing fluorescence of the fluorophore. Hence, fluorescence detected in the quantitative PCR thermal cycler is directly proportional to the fluorophore released and the amount of DNA template present in the PCR.

Primers and probes are generally between 5 and 100 nucleotides or more in length. In some embodiments, primers and probes can be at least 20 nucleotides or more in length, or at least 25 nucleotides or more, or at least 30 nucleotides or more in length. Such primers and probes hybridize specifically to a target sequence under optimum hybridization conditions as are known in the art. Primers and probes according to the present invention may have complete sequence complementarity with the target sequence, although probes differing from the target sequence and which retain the ability to hybridize to target sequences may be designed by conventional methods according to the invention.

Methods for preparing and using probes and primers are described, for example, in Molecular Cloning: A Laboratory Manual, 2nd ed., vol. 1-3, ed. Sambrook et al., Cold Spring Harbor Laboratory Press, Cold Spring Harbor, N.Y., 1989. PCR-primer pairs can be derived from a known sequence, for example, by using computer programs intended for that purpose.

The polymerase chain reaction (PCR) is a technique for “amplifying” a particular piece of DNA. In order to perform PCR, at least a portion of the nucleotide sequence of the DNA molecule to be replicated must be known. In general, primers or short oligonucleotides are used that are complementary (e.g., substantially complementary or fully complementary) to the nucleotide sequence at the 3′ end of each strand of the DNA to be amplified (known sequence). The DNA sample is heated to separate its strands and is mixed with the primers. The primers hybridize to their complementary sequences in the DNA sample. Synthesis begins (5′ to 3′ direction) using the original DNA strand as the template. The reaction mixture must contain all four deoxynucleotide triphosphates (dATP, dCTP, dGTP and dTTP) and a DNA polymerase. Polymerization continues until each newly-synthesized strand has proceeded far enough to contain the sequence recognized by the other primer. Once this occurs, two DNA molecules are created that are identical to the original molecule. These two molecules are heated to separate their strands and the process is repeated. Each cycle doubles the number of DNA molecules. Using automated equipment, each cycle of replication can be completed in less than 5 minutes. After 30 cycles, what began as a single molecule of DNA has been amplified into more than a billion copies (2³⁰=1.02×10⁹).

The oligonucleotides of an oligonucleotide primer pair are complementary to DNA sequences located on opposite DNA strands and flanking the region to be amplified. The annealed primers hybridize to the newly synthesized DNA strands. The first amplification cycle will result in two new DNA strands whose 5′ end is fixed by the position of the oligonucleotide primer but whose 3′ end is variable (‘ragged’ 3′ ends). The two new strands can serve in turn as templates for synthesis of complementary strands of the desired length (the 5′ ends are defined by the primer and the 3′ ends are fixed because synthesis cannot proceed past the terminus of the opposing primer). After a few cycles, the desired fixed length product begins to predominate.

A quantitative polymerase chain reaction (qPCR), also referred to as real-time polymerase chain reaction, monitors the accumulation of a DNA product from a PCR reaction in real time. qPCR is a laboratory technique of molecular biology based on the polymerase chain reaction (PCR), which is used to amplify and simultaneously quantify a targeted DNA molecule. Even one copy of a specific sequence can be amplified and detected in PCR. The PCR reaction generates copies of a DNA template exponentially. This results in a quantitative relationship between the amount of starting target sequence and amount of PCR product accumulated at any particular cycle. Due to inhibitors of the polymerase reaction found with the template, reagent limitation or accumulation of pyrophosphate molecules, the PCR reaction eventually ceases to generate template at an exponential rate (i.e., the plateau phase), making the end point quantitation of PCR products unreliable. Therefore, duplicate reactions may generate variable amounts of PCR product. Only during the exponential phase of the PCR reaction is it possible to extrapolate back in order to determine the starting quantity of template sequence. The measurement of PCR products as they accumulate (i.e., real-time quantitative PCR) allows quantitation in the exponential phase of the reaction and therefore removes the variability associated with conventional PCR. In a real time PCR assay, a positive reaction is detected by accumulation of a fluorescent signal. For one or more specific sequences in a DNA sample, quantitative PCR enables both detection and quantification. The quantity can be either an absolute number of copies or a relative amount when normalized to DNA input or additional normalizing genes. Since the first documentation of real-time PCR, it has been used for an increasing and diverse number of applications including mRNA expression studies, DNA copy number measurements in genomic or viral DNAs, allelic discrimination assays, expression analysis of specific splice variants of genes and gene expression in paraffin-embedded tissues and laser captured micro-dissected cells.

As used herein, the phrase “Ct value” refers to “threshold cycle,” which is defined as the “fractional cycle number at which the amount of amplified target reaches a fixed threshold.” In some embodiments, it represents an intersection between an amplification curve and a threshold line. The amplification curve is typically in an “S” shape indicating the change of relative fluorescence of each reaction (Y-axis) at a given cycle (X-axis), which in some embodiments is recorded during PCR by a real-time PCR instrument. The threshold line is in some embodiments the level of detection at which a reaction reaches a fluorescence intensity above background. See Livak & Schmittgen (2001) 25 Methods 402-408. It is a relative measure of the concentration of the target in the PCR. Generally, good Ct values for quantitative assays such as qPCR are in some embodiments in the range of 10-40 for a given reference gene. Ct levels are inversely proportional to the amount of target nucleic acid in the sample (i.e. the lower the Ct level the greater the amount of detectable target nucleic acid in the sample). Additionally, good Ct values for quantitative assays such as qPCR show a linear response range with proportional dilutions of target gDNA.

In some embodiments, qPCR is performed under conditions wherein the Ct value can be collected in real-time for quantitative analysis. For example, in a typical qPCR experiment, DNA amplification is monitored at each cycle of PCR during the extension stage. The amount of fluorescence generally increases above the background when DNA is in the log linear phase of amplification. In some embodiments, the Ct value is collected at this time point.

As used herein, the term “cell” refers to any living cell. The cell may be a prokaryotic or eukaryotic cell. The cell may be isolated. The cell may or may not be capable of regenerating into an organism. The cell may be in the context of a tissue, callus, culture, organ, or part. In some embodiments, the cell may be a plant cell. A plant cell of the present invention can be in the form of an isolated single cell or can be a cultured cell or can be a part of a higher-organized unit such as, for example, a plant tissue or a plant organ. The plant cell may be derived from or part of an angiosperm or gymnosperm. In further embodiments, the plant cell may be a monocotyledonous plant cell, a dicotyledonous plant cell. The monocotyledonous plant cell may be, for example, a maize, rice, sorghum, sugarcane, barley, wheat, oat, turf grass, or ornamental grass cell. The dicotyledonous plant cell may be, for example, a tobacco, pepper, eggplant, sunflower, crucifer, flax, potato, cotton, soybean, sugar bee, or oilseed rape cell.

The term “plant part,” as used herein, includes but is not limited to embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast.

The term “introducing” or “introduce” in the context of a cell, prokaryotic cell, bacterial cell, eukaryotic cell, plant cell, plant and/or plant part means contacting a nucleic acid molecule with the cell, eukaryotic cell, plant, plant part, and/or plant cell in such a manner that the nucleic acid molecule gains access to the interior of the cell, eukaryotic cell, plant cell and/or a cell of the plant and/or plant part. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol.

An “inversion” is a chromosome rearrangement in which a segment of a chromosome is reversed end to end. An inversion occurs when a single chromosome undergoes breakage and rearrangement within itself. A chromosome “translocation” is a rearrangement of parts between non-homologous chromosomes.

As used herein, the terms “transformed” and “transgenic” refer to any cell, prokaryotic cell, eukaryotic cell, plant, plant cell, callus, plant tissue, or plant part that contains all or part of at least one recombinant (e.g., heterologous) polynucleotide. In some embodiments, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

The term “transformation” as used herein refers to the introduction of a heterologous nucleic acid into a cell. Transformation of a cell may be stable or transient. Thus, a transgenic cell, plant cell, plant and/or plant part of the invention can be stably transformed or transiently transformed. Transformation can refer to the transfer of a nucleic acid molecule into the genome of a host cell, resulting in genetically stable inheritance. In some embodiments, the introduction into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of nucleic acid into the plant, plant part and/or cell thereof, or any combination thereof.

Procedures for transforming plants are well known and routine in the art and are described throughout the literature. Non-limiting examples of methods for transformation of plants include transformation via bacterial-mediated nucleic acid delivery (e.g. via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell Mol Biol Lett 7:849-858 (2002)).

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. 1993, Plant Cell 5:159-169). The transfer of the recombinant binary vector to Agrobacterium can be accomplished by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hagen and Willmitzer 1988, Nucleic Acids Res 16:9877).

Transformation of a plant by recombinant Agrobacterium usually involves co-cultivation of the Agrobacterium with explants from the plant and follows methods well known in the art. Transformed tissue is typically regenerated on selection medium carrying an antibiotic or herbicide resistance marker between the binary plasmid T-DNA borders.

Another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacteria or a bacteriophage, each containing one or more nucleic acids sought to be introduced) also can be propelled into plant tissue.

“Transient transformation” in the context of a polynucleotide means that a polynucleotide is introduced into the cell and does not integrate into the genome of the cell.

As used herein, “stably introducing,” “stably introduced,” “stable transformation” or “stably transformed” in the context of a polynucleotide introduced into a cell, means that the introduced polynucleotide is stably integrated into the genome of the cell, and thus the cell is stably transformed with the polynucleotide. As such, the integrated polynucleotide is capable of being inherited by the progeny thereof, more particularly, by the progeny of multiple successive generations. “Genome” as used herein includes the nuclear and/or plastid genome, and therefore includes integration of a polynucleotide into, for example, the chloroplast genome. Stable transformation as used herein can also refer to a polynucleotide that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more nucleic acid molecules introduced into an organism. Stable transformation of a cell can be detected by, for example, a Southern blot hybridization assay of genomic DNA of the cell with nucleic acid sequences which specifically hybridize with a nucleotide sequence of a nucleic acid molecule introduced into an organism (e.g., a plant). Stable transformation of a cell can be detected by, for example, a Northern blot hybridization assay of RNA of the cell with nucleic acid sequences which specifically hybridize with a nucleotide sequence of a nucleic acid molecule introduced into a plant or other organism. Stable transformation of a cell can also be detected by, e.g., a polymerase chain reaction (PCR) or other amplification reaction as are well known in the art, employing specific primer sequences that hybridize with target sequence(s) of a nucleic acid molecule, resulting in amplification of the target sequence(s), which can be detected according to standard methods. Transformation can also be detected by direct sequencing and/or hybridization protocols well known in the art.

Thus, in particular embodiments of the present invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

The “transformation and regeneration process” refers to the process of stably introducing a transgene into a plant cell and regenerating a plant from the transgenic plant cell. As used herein, transformation and regeneration includes the selection process, whereby a transgene comprises a selectable marker and the transformed cell has incorporated and expressed the transgene, such that the transformed cell will survive and developmentally flourish in the presence of the selection agent. “Regeneration” refers to growing a whole plant from a plant cell, a group of plant cells, or a plant piece such as from a protoplast, callus, or tissue part.

The terms “nucleotide sequence” “nucleic acid,” “nucleic acid sequence,” “nucleic acid molecule,” “oligonucleotide” and “polynucleotide” are used interchangeably herein to refer to a heteropolymer of nucleotides and encompass both RNA and DNA, including cDNA, genomic DNA, mRNA, synthetic (e.g., chemically synthesized) DNA or RNA and chimeras of RNA and DNA. The term nucleic acid molecule refers to a chain of nucleotides without regard to length of the chain. The nucleotides contain a sugar, phosphate and a base which is either a purine or pyrimidine. A nucleic acid molecule can be double-stranded or single-stranded. Where single-stranded, the nucleic acid molecule can be a sense strand or an antisense strand. A nucleic acid molecule can be synthesized using oligonucleotide analogs or derivatives (e.g., inosine or phosphorothioate nucleotides). Such oligonucleotides can be used, for example, to prepare nucleic acid molecules that have altered base-pairing abilities or increased resistance to nucleases. Nucleic acid sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25.

A “nucleic acid fragment” is a fraction of a given nucleic acid molecule. An “RNA fragment” is a fraction of a given RNA molecule. A “DNA fragment” is a fraction of a given DNA molecule. A “nucleic acid segment” is a fraction of a given nucleic acid molecule and is not isolated from the molecule. An “RNA segment” is a fraction of a given RNA molecule and is not isolated from the molecule. A “DNA segment” is a fraction of a given DNA molecule and is not isolated from the molecule. Segments of polynucleotides can be any length, for example, at least 5, 10, 15, 20, 25, 30, 40, 50, 75, 100, 150, 200, 300 or 500 or more nucleotides in length. A segment or portion of a guide sequence can be about 50%, 40%, 30%, 20%, 10% of the guide sequence, e.g., one-third of the guide sequence or shorter, e.g., 7, 6, 5, 4, 3, or 2 nucleotides in length.

The term “derived from” in the context of a molecule refers to a molecule isolated or made using a parent molecule or information from that parent molecule. For example, a Cas9 single mutant nickase and a Cas9 double mutant null-nuclease are derived from a wild-type Cas9 protein.

In higher plants, deoxyribonucleic acid (DNA) is the genetic material while ribonucleic acid (RNA) is involved in the transfer of information contained within DNA into proteins. A “genome” is the entire body of genetic material contained in each cell of an organism. Unless otherwise indicated, a particular nucleic acid sequence of this invention also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid molecule is used interchangeably with gene, cDNA, and mRNA encoded by a gene.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, the phrase “substantially identical,” in the context of two nucleic acid molecules, nucleotide sequences or protein sequences, refers to two or more sequences or subsequences that have at least about 70%, least about 75%, at least about 80%, least about 85%, at least about 90%, at least about 95%, at least about 96%, at least about 97%, at least about 98%, or at least about 99% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 50 residues to about 150 residues in length. Thus, in some embodiments of this invention, the substantial identity exists over a region of the sequences that is at least about 50, about 60, about 70, about 80, about 90, about 100, about 110, about 120, about 130, about 140, about 150, or more residues in length. In some particular embodiments, the sequences are substantially identical over at least about 150 residues. In a further embodiment, the sequences are substantially identical over the entire length of the coding regions. Furthermore, in representative embodiments, substantially identical nucleotide or protein sequences perform substantially the same function (e.g., guiding to a particular genomic target, endonuclease cleavage of a particular genomic target site).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., 1990). These initial neighbourhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences can also be considered to be substantially identical when the two sequences hybridize to each other under stringent conditions. In some representative embodiments, two nucleotide sequences considered to be substantially identical hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the present invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2×SSC, 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1×SSC, 0.1% SDS at 50° C. or in 7% sodium dodecyl sulfate (SDS), 0.5 M NaPO₄, 1 mM EDTA at 50° C. with washing in 0.5×SSC, 0.1% SDS at 50° C. In still further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1×SSC, 0.1% SDS at 50° C., or in 7% sodium dodecyl sulfate (SDS), 0.5 M NaPO₄, 1 mM EDTA at 50° C. with washing in 0.1×SSC, 0.1% SDS at 65° C.

An “isolated” nucleic acid molecule or nucleotide sequence or an “isolated” polypeptide is a nucleic acid molecule, nucleotide sequence or polypeptide that, by the hand of man, exists apart from its native environment and/or has a function that is different, modified, modulated and/or altered as compared to its function in its native environment and is therefore not a product of nature. An isolated nucleic acid molecule or isolated polypeptide may exist in a purified form or may exist in a non-native environment such as, for example, a recombinant host cell. Thus, for example, with respect to polynucleotides, the term isolated means that it is separated from the chromosome and/or cell in which it naturally occurs. A polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and nucleotide sequences of the invention can be considered to be “isolated” as defined above.

Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid molecule encoding an additional polypeptide or peptide sequence. An “isolated nucleic acid molecule” or “isolated nucleotide sequence” can also include a nucleotide sequence derived from and inserted into the same natural, original cell type, but which is present in a non-natural state, e.g., present in a different copy number, and/or under the control of different regulatory sequences than that found in the native state of the nucleic acid molecule.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not necessarily mean that the preparation is technically pure (homogeneous), but it is sufficiently pure to provide the polypeptide or nucleic acid in a form in which it can be used for the intended purpose.

In representative embodiments of the invention, an “isolated” nucleic acid molecule, nucleotide sequence, and/or polypeptide is at least about 5%, 10%, 15%, 20%, 25%, 30%, 40%, 50%, 60%, 70%, 75%, 80%, 85%, 90%, 95%, 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10,000-fold, 100,000-fold or more enrichment of the nucleic acid (w/w) is achieved as compared with the starting material.

“Wild-type” nucleotide sequence or amino acid sequence refers to a naturally occurring (“native”) or endogenous nucleotide sequence or amino acid sequence. Thus, for example, a “wild-type mRNA” is an mRNA that is naturally occurring in or endogenous to the organism. A “homologous” nucleotide sequence is a nucleotide sequence naturally associated with a host cell into which it is introduced.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Promoter” refers to a nucleotide sequence, usually upstream (5′) to its coding sequence, which controls the expression of the coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. The meaning of the term “promoter” includes “promoter regulatory sequences.”

“Primary transformant” and “T0 generation” refer to transgenic plants that are of the same genetic generation as the tissue that was initially transformed (i.e., not having gone through meiosis and fertilization since transformation). “Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

A “transgene” refers to a nucleic acid molecule that has been introduced into the genome by transformation and is stably maintained. A transgene may comprise at least one expression cassette, typically comprises at least two expression cassettes, and may comprise ten or more expression cassettes. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but one that is introduced into the organism by gene transfer.

“Intron” refers to an intervening section of DNA which occurs almost exclusively within a eukaryotic gene, but which is not translated to amino acid sequences in the gene product. The introns are removed from the pre-mature mRNA through a process called splicing, which leaves the exons untouched, to form an mRNA. For purposes of the present invention, the definition of the term “intron” includes modifications to the nucleotide sequence of an intron derived from a target gene, provided the modified intron does not significantly reduce the activity of its associated 5′ regulatory sequence.

“Exon” refers to a section of DNA which carries the coding sequence for a protein or part of it. Exons are separated by intervening, non-coding sequences (introns). For purposes of the present invention, the definition of the term “exon” includes modifications to the nucleotide sequence of an exon derived from a target gene, provided the modified exon does not significantly reduce the activity of its associated 5′ regulatory sequence.

The term “cleavage” or “cleaving” refers to breaking of the covalent phosphodiester linkage in the ribosylphosphodiester backbone of a polynucleotide. The terms “cleavage” or “cleaving” encompass both single-stranded breaks and double-stranded breaks. Double-stranded cleavage can occur as a result of two distinct single-stranded cleavage events. Cleavage can result in the production of either blunt ends or staggered ends. A “nuclease cleavage site” or “genomic nuclease cleavage site” is a region of nucleotides that comprise a nuclease cleavage sequence that is recognized by a specific nuclease, which acts to cleave the nucleotide sequence of the genomic DNA in one or both strands. Such cleavage by the nuclease enzyme initiates DNA repair mechanisms within the cell, which establishes an environment for homologous recombination to occur.

A “donor molecule” or “donor sequence” is a nucleotide polymer or oligomer intended for insertion at a target polynucleotide, typically a target genomic site. The donor sequence may be one or more transgenes, expression cassettes, or nucleotide sequences of interest. A donor molecule may be a donor DNA molecule, either single stranded, partially double-stranded, or double-stranded. The donor polynucleotide may be a natural or a modified polynucleotide, a RNA-DNA chimera, or a DNA fragment, either single- or at least partially double-stranded, or a fully double-stranded DNA molecule, or a PGR amplified ssDNA or at least partially dsDNA fragment. In some embodiments, the donor DNA molecule is part of a circularized DNA molecule. A fully double-stranded donor DNA is advantageous since it might provide an increased stability, since dsDNA fragments are generally more resistant than ssDNA to nuclease degradation. In some embodiments, the donor polynucleotide molecule can comprise at least about 100, 150, 200, 250, 300, 250, 400, 450, 500, 600, 700, 800, 900, 1000, 1500, 2000, 2500, 3000, 3500, 4000, 4500, 5000, 7500, 10000, 15,000 or 20,000 nucleotides, including any value within this range not explicitly recited herein. In some embodiments, the donor DNA molecule comprises heterologous nucleic acid sequence. In some embodiments, the donor DNA molecule comprises at least one expression cassette. In some embodiments, the donor DNA molecule may comprise a transgene, which comprises at least one expression cassette. In some embodiments, the donor DNA molecule comprises an allelic modification of a gene which is native to the target genome. The allelic modification may comprise at least one nucleotide insertion, at least one nucleotide deletion, and/or at least one nucleotide substitution. In some embodiments, the allelic modification may comprise an INDEL. In some embodiments, the donor DNA molecule comprises homologous arms to the target genomic site. In some embodiments, the donor DNA molecule comprises at least 100 contiguous nucleotides at least 90% identical to a genomic nucleic acid sequence, and optionally may further comprise a heterologous nucleic acid sequence such as a transgene.

As used herein, the terms “proximal” or “proximal to” with regard to one or more nucleotide sequences of this invention means immediately next to (e.g., with no intervening sequence) or separated by from about 1 base to about 500 bases (e.g., 1, 2, 3, 4, 5, 6, 7, 8, 9, 10, 15, 20, 30, 40, 50, 100, 200, 250, 300, 350, 400, 450, or 500 bases), including any values included within this range but not explicitly recited herein.

As used herein, the term “guide RNA” or “gRNA” generally refers to an RNA molecule (or a group of RNA molecules collectively) that can bind to a CRISPR system effector, such as a Cas or a Cpf1 protein, and aid in targeting the Cas or Cpf1 protein to a specific location within a target polynucleotide (e.g., a DNA). A guide RNA of the invention can be an engineered, single RNA molecule (sgRNA), where for example the sgRNA comprises a crRNA segment and optionally a tracrRNA segment. A guide RNA of the invention can also be a dual-guide system, where the crRNA and tracrRNA molecules are physically distinct molecules which then interact to form a duplex for recruitment of a CRISPR system effector, such as Cas9, and for targeting of that protein to the target polynucleotide.

As used herein, the term “crRNA” or “crRNA segment” refers to an RNA molecule or to a portion of an RNA molecule that includes a polynucleotide targeting guide sequence, a stem sequence involved in protein-binding, and, optionally, a 3′-overhang sequence. The polynucleotide targeting guide sequence is a nucleic acid sequence that is complementary to a sequence in a target DNA. This polynucleotide targeting guide sequence is also referred to as the “protospacer”. In other words, the polynucleotide targeting guide sequence of a crRNA molecule interacts with a target DNA in a sequence-specific manner via hybridization (i.e., base pairing). As such, the nucleotide sequence of the polynucleotide targeting guide sequence of the crRNA molecule may vary and determines the location within the target DNA that the guide RNA and the target DNA will interact.

The polynucleotide targeting guide sequence of a crRNA molecule can be modified (e.g., by genetic engineering) to hybridize to any desired sequence within a target DNA. The polynucleotide targeting guide sequence of a crRNA molecule of the invention can have a length from about 12 nucleotides to about 100 nucleotides. For example, the polynucleotide targeting guide sequence of a crRNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the polynucleotide targeting guide sequence of a crRNA can have a length of from about 17 nt to about 27 nts. For example, the polynucleotide targeting guide sequence of a crRNA can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The nucleotide sequence of the polynucleotide targeting guide sequence of a crRNA can have a length at least about 12 nt. In some embodiments, the polynucleotide targeting guide sequence of a crRNA is 20 nucleotides in length. In some embodiments, the polynucleotide targeting guide sequence of a crRNA is 19 nucleotides in length.

The present invention also provides a guide RNA comprising an engineered crRNA, wherein the crRNA comprises a bait RNA segment capable of hybridizing to a genomic target sequence. This engineered crRNA maybe a physically distinct molecule, as in a dual-guide system.

As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The present invention also provides a guide RNA comprising an engineered tracrRNA, wherein the tracrRNA further comprises a bait RNA segment that is capable of binding to a donor DNA molecule. The engineered tracrRNA may be a physically distinct molecule, as in a dual-guide system, or may be a segment of a sgRNA molecule.

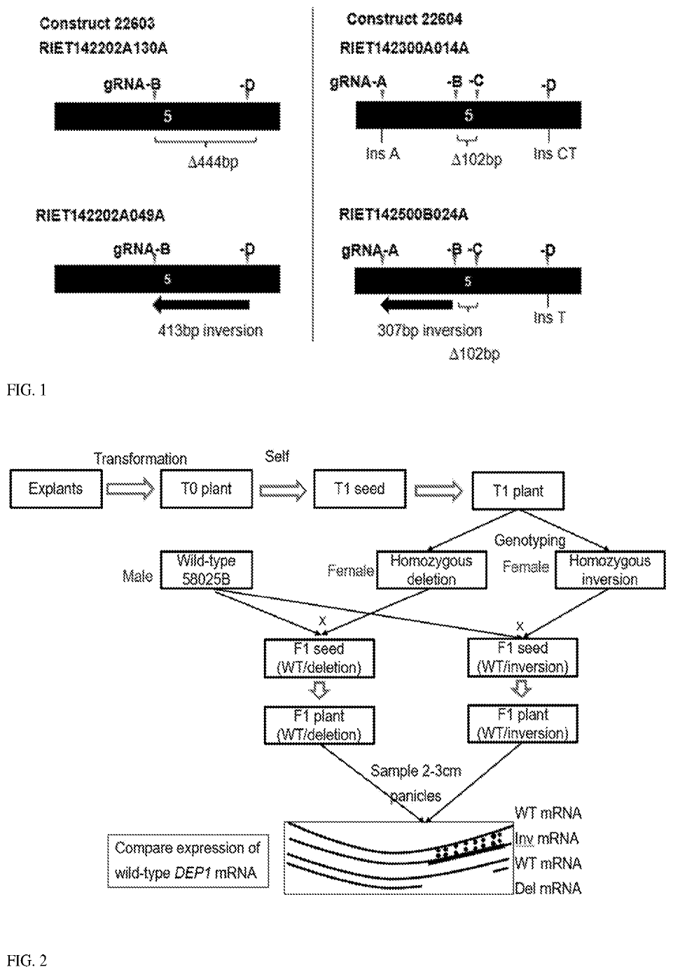

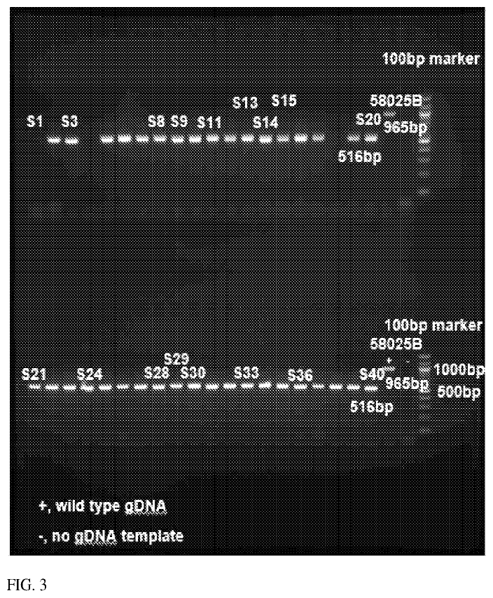

In some embodiments, the guide RNA, either as a sgRNA or as two or more RNA molecules, does not contain a tracrRNA, as it is known in the art that some CRISPR-associated nucleases, such as Cpf1 (also known as Cas12a), do not require a tracrRNA for its RNA-mediated endonuclease activity (Qi et al., 2013, Cell, 152: 1173-1183; Zetsche et al., 2015, Cell 163: 759-771). Such a guide RNA of the invention may comprise a crRNA with the bait RNA operably linked at the 5′ or 3′ end of the crRNA. Cpf1 also has RNase activity on its cognate pre-crRNA (Fonfara et al., 2016, Nature, doi.org/10.1038/nature17945). A guide RNA of the invention may comprise multiple crRNAs which the Cpf1 possesses to mature crRNAs. In some embodiments, each of these crRNAs is operably linked to a bait RNA. In other embodiments, at least one of these crRNAs is operably linked to a bait RNA. The bait RNA may be specific to a sequence of interest (SOI), as shown in FIG. 1 and as described in the Examples herein, or it may be a “universal” bait, which has a corresponding “universal” prey sequence on the donor DNA molecule, as shown in FIG. 2 and as described in the Examples herein.

The present invention also provides a nucleic acid molecule comprising a nucleic acid sequence encoding a guide RNA of the invention. The nucleic acid molecule may be a DNA or an RNA molecule. In some embodiments, the nucleic acid molecule is circularized. In other embodiments, the nucleic acid molecule is linear. In some embodiments, the nucleic acid molecule is single stranded, partially double-stranded, or double-stranded. In some embodiments, the nucleic acid molecule is complexed with at least one polypeptide. The polypeptide may have a nucleic acid recognition or nucleic acid binding domain. In some embodiments, the polypeptide is a shuttle for mediating delivery of, for example, a chimeric RNA of the invention, a nuclease, and optionally a donor molecule. In some embodiments, the polypeptide is a Feldan Shuttle (U.S. Patent Publication No. 20160298078, herein incorporated by reference). The nucleic acid molecule may comprise an expression cassette capable of driving the expression of the chimeric RNA. The nucleic acid molecule may further comprise additional expression cassettes, capable of expressing, for example, a nuclease such as a CRISPR-associated nuclease. The present invention also provides an expression cassette comprising a nucleic acid sequence encoding a chimeric RNA of the invention.

A “site-directed modifying polypeptide” modifies the target DNA (e.g., cleavage or methylation of target DNA) and/or a polypeptide associated with target DNA (e.g., methylation or acetylation of a histone tail). A site-directed modifying polypeptide is also referred to herein as a “site-directed polypeptide” or an “RNA binding site-directed modifying polypeptide.” The site-directed modifying polypeptide interacts with the guide RNA, which is either a single RNA molecule or a RNA duplex of at least two RNA molecules, and is guided to a DNA sequence (e.g. a chromosomal sequence or an extrachromosomal sequence, e.g. an episomal sequence, a minicircle sequence, a mitochondrial sequence, a chloroplast sequence, etc.) by virtue of its association with the guide RNA.

In some cases, the site-directed modifying polypeptide is a naturally-occurring modifying polypeptide. In other cases, the site-directed modifying polypeptide is not a naturally-occurring polypeptide (e.g., a chimeric polypeptide or a naturally-occurring polypeptide that is modified, e.g., mutation, deletion, insertion). Exemplary naturally-occurring site-directed modifying polypeptides are known in the art (see for example, Makarova et al., 2017, Cell 168: 328-328.e1, and Shmakov et al., 2017, Nat Rev Microbiol 15(3): 169-182, both herein incorporated by reference). These naturally occurring polypeptides bind a DNA-targeting RNA, are thereby directed to a specific sequence within a target DNA, and cleave the target DNA to generate a double strand break.

A site-directed modifying polypeptide comprises two portions, an RNA-binding portion and an activity portion. In some embodiments, the site-directed modifying polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that exhibits site-directed enzymatic activity (e.g., activity for DNA methylation, activity for DNA cleavage, activity for histone acetylation, activity for histone methylation, etc.), wherein the site of enzymatic activity is determined by the DNA-targeting RNA. In other embodiments, a site-directed modifying polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that modulates transcription within the target DNA (e.g., to increase or decrease transcription), wherein the site of modulated transcription within the target DNA is determined by the DNA-targeting RNA.

In some cases, the site-directed modifying polypeptide has enzymatic activity that modifies target DNA (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). In other cases, the site-directed modifying polypeptide has enzymatic activity that modifies a polypeptide (e.g., a histone) associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

In some cases, different site-directed modifying polypeptides, for example different Cas9 proteins (i.e., Cas9 proteins from various species) may be advantageous to use in the various provided methods of the invention to capitalize on various enzymatic characteristics of the different Cas9 proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.). Cas9 proteins from various species (for example, those disclosed in Shmakov et al., 2017, or polypeptides derived therefrom) may require different PAM sequences in the target DNA. Thus, for a particular Cas9 enzyme of choice, the PAM sequence requirement may be different than the 5′-N GG-3′ sequence (where N is either a A, T, C, or G) known to be required for Cas9 activity. Many Cas9 orthologues from a wide variety of species have been identified herein and the proteins share only a few identical amino acids. All identified Cas9 orthologs have the same domain architecture with a central HNH endonuclease domain and a split RuvC/RNaseH domain. Cas9 proteins share 4 key motifs with a conserved architecture; Motifs 1, 2, and 4 are RuvC like motifs, while motif 3 is an HNH-motif.

The site-directed modifying polypeptide may also be a chimeric and modified Cas9 nuclease. For example, it may be a modified Cas9 “base editor”. Base editing enables direct, irreversible conversion of one target DNA base into another in a programmable manner, without requiring DNA cleavage or a donor DNA molecule. For example, Komor et al (2016, Nature, 533: 420-424), teach a Cas9-cytidine deaminase fusion, where the Cas9 has also been engineered to be inactivated and not induce double-stranded DNA breaks. Additionally, Gaudelli et al (2017, Nature, doi:10.1038/nature24644) teach a catalytically impaired Cas9 fused to a tRNA adenosine deaminase, which can mediate conversion of an A/T to G/C in a target DNA sequence. Another class of engineered Cas9 nucleases which may act as a site-directed modifying polypeptide in the methods and compositions of the invention are variants which can recognize a broad range of PAM sequences, including NG, GAA, and GAT (Hu et al., 2018, Nature, doi:10.1038/nature26155).

Any Cas9 protein, including those naturally occurring and/or those mutated or modified from naturally occurring Cas9 proteins, can be used as a site-directed modifying polypeptide in the methods and compositions of the present invention. Catalytically active Cas9 nucleases cleave target DNA to produce double strand breaks. These breaks are then repaired by the cell in one of two ways: non-homologous end joining, and homology-directed repair.

In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor DNA molecule with homology to the cleaved target DNA sequence is used as a template for repair of the cleaved target DNA sequence, resulting in the transfer of genetic information from the donor polynucleotide to the target DNA. As such, new nucleic acid material may be inserted/copied into the site. In some cases, a target DNA is contacted with a donor molecule, for example a donor DNA molecule. In some cases, a donor DNA molecule is introduced into a cell. In some cases, at least a segment of a donor DNA molecule integrates into the genome of the cell.

The modifications of the target DNA due to NHEJ and/or homology-directed repair lead to, for example, gene correction, gene replacement, gene tagging, transgene insertion, nucleotide deletion, gene disruption, gene mutation, etc. Accordingly, cleavage of DNA by a site-directed modifying polypeptide may be used to delete nucleic acid material from a target DNA sequence (e.g., to disrupt a gene that makes cells susceptible to infection (e.g. the CCR5 or CXCR4 gene, which makes T cells susceptible to HIV infection), to remove disease-causing trinucleotide repeat sequences in neurons, to create gene knockouts and mutations as disease models in research, etc.) by cleaving the target DNA sequence and allowing the cell to repair the sequence in the absence of an exogenously provided donor polynucleotide. Thus, the subject methods can be used to knock out a gene (resulting in complete lack of transcription or altered transcription) or to knock in genetic material into a locus of choice in the target DNA. Alternatively, if a DNA-targeting RNA duplex and a site-directed modifying polypeptide are co-administered to cells with a donor molecule that includes at least a segment with homology to the target DNA sequence, the subject methods may be used to add, i.e. insert or replace, nucleic acid material to a target DNA sequence (e.g. to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, a complex comprising a DNA-targeting RNA duplex and a site-directed modifying polypeptide is useful in any in vitro or in vivo application in which it is desirable to modify DNA in a site-specific, i.e. “targeted”, way, for example gene knock-out, gene knock-in, gene editing, gene tagging, etc., as used in, for example, gene therapy, e.g. to treat a disease or as an antiviral, antipathogenic, or anticancer therapeutic, the production of genetically modified organisms in agriculture, the large scale production of proteins by cells for therapeutic, diagnostic, or research purposes, the induction of iPS cells, biological research, the targeting of genes of pathogens for deletion or replacement, etc.

The term “CRISPR-associated protein”, “Cas protein”, “CRISPR-associated nuclease” or “Cas nuclease” refers to a wild type Cas protein, a fragment thereof, or a mutant or variant thereof. The term “Cas mutant” or “Cas variant” refers to a protein or polypeptide derivative of a wild type Cas protein, e.g., a protein having one or more point mutations, insertions, deletions, truncations, a fusion protein, or a combination thereof. In certain embodiments, the Cas mutant or Cas variant substantially retains the nuclease activity of the Cas protein, such as a Cas9 variant described herein which is operably linked to a nuclear localization signal (NLS) derived from a plant. In certain embodiments, the Cas nuclease is mutated such that one or both nuclease domains are inactive, such as, for example, a catalytically dead Cas9 referred to as dCas9, which is still able to target to a specific genomic location but has no endonuclease activity (Qi et al., 2013, Cell, 152: 1173-1183, hereby incorporated within). In some embodiments, the Cas nuclease is mutated so that it lacks some or all of the nuclease activity of its wild-type counterpart. The Cas protein may be Cas9, Cpf1 (Zetsche et al., 2015, Cell, 163: 759-771, hereby incorporated within) or any another CRISPR-associated nuclease.
a. The present invention provides a method of gene silencing a target gene comprised of: introducing into a cell a nuclease capable of site-directed DNA cleavage at a target genomic site, making two or more double strand cuts within a single target gene, selecting for a cell where the double strand cuts have been repaired with the intervening DNA inverted, and silencing expression of the target gene.

In some embodiments, the invention provides the methods described above, further comprising introducing into the cell a third nucleic acid molecule comprising a nucleotide sequence encoding an anti-silencing polypeptide. In some embodiments, the anti-silencing polypeptide may be provided to the cell. In some embodiments, the anti-silencing protein is or is derived from a viral silencing suppressor (VSR). In further embodiments, the anti-silencing protein is a VSR derived from a plant virus. In further embodiments, the anti-silencing protein is the viral silencing suppressor p19 protein, derived from a Tombus virus, for example CymRSV, CIRV, or TBSV. Zhu et al recently showed that p19 VSR derived from Tomato Bushy Stunt Virus co-expressed with a guide RNA and a Cas9 nuclease improved gene targeting efficiency and/or guide RNA stability in plants (U.S. Patent Publication No. 2016/0264982). In some embodiments, the VSR is selected from the group of plant virus proteins including HC-Pro, p14, p38, NSs, NS3, CaMV P6, PNS10, P122, 2b, Potex p25, ToRSV CP, P0, and SPMMV P1 (see Csorba et al., 2015, Virology 479-480 p. 85-103, hereby incorporated by reference).

In some embodiments, the invention provides the methods described above where the second nucleic acid molecule encodes a site-directed modifying polypeptide. In further embodiments, the site-directed modifying polypeptide is a nuclease. In still further embodiments, the site-directed modifying polypeptide is a nuclease that is an endonuclease, for example a meganuclease, a zinc finger nuclease, or a TALEN. In some embodiments, the nuclease is an RNA-guided endonuclease. In further embodiments, the nuclease is a CRISPR-associated nuclease, for example Cas9 or Cpf1 or a mutant variant of Cas9 or Cpf1, for example a nuclease-deactivated mutant variant, or a fusion between at least one domain of Cas9 or Cpf1 and at least one domain of a different site-directed modifying polypeptide.

In some embodiments, the invention provides the methods described above, further comprising introducing into the cell a third nucleic acid molecule comprising a nucleotide sequence encoding an anti-silencing polypeptide. In some embodiments, the anti-silencing protein is or is derived from a viral silencing suppressor (VSR). In further embodiments, the anti-silencing protein is a VSR derived from a plant virus. In further embodiments, the anti-silencing protein is the viral silencing suppressor p19 protein, derived from a Tombus virus, for example CymRSV, CIRV, or TBSV. Zhu et al recently showed that p19 VSR derived from Tomato Bushy Stunt Virus co-expressed with a guide RNA and a Cas9 nuclease improved gene targeting efficiency and/or guide RNA stability in plants (U.S. Patent Publication No. 2016/0264982). In some embodiments, the VSR is selected from the group of plant virus proteins including HC-Pro, p14, p38, NSs, NS3, CaMV P6, PNS10, P122, 2b, Potex p25, ToRSV CP, P0, and SPMMV P1 (see Csorba et al., 2015, Virology 479-480 p. 85-103, hereby incorporated by reference).

The present disclosure provides a method of reducing expression a target gene comprised of, introducing into a cell a nuclease capable of site-directed DNA cleavage at a target genomic site making two or more double strand cuts within a single target gene, selecting for a cell where the double strand cuts have been repaired with the intervening DNA inverted, and reducing expression of the target gene. In some embodiments, the nuclease is selected from the group consisting of meganucleases (MNs), zinc-finger nucleases (ZFNs), transcription-activator like effector nucleases (TALENs), Cas9 nuclease, Cfp1 nuclease, dCas9-FokI, dCpf1-FokI, chimeric Cas9/Cpf1-cytidine deaminase, chimeric Cas9/Cpf1-adenine deaminase, chimeric FEN1-FokI, and Mega-TALs, a nickase Cas9 (nCas9), chimeric dCas9 non-FokI nuclease and dCpf1 non-FokI nuclease. In some embodiments of the method, the double strand cuts in the target gene are located in the promoter, UTR, exon, intron, or gene-gene junction region. These methods may be used when the cell has a haploid, diploid, polyploid, or hexiploid genome. These methods may be used when the target gene is dominant, recessive, or semi-dominant. In some embodiments the method may make use of one, two or more guide sequences. This method is useful in plant cells, but is applicable to any cell.

The present disclosure provides a method of rearranging a chromosome by genome editing comprising generating at least one breakage in the chromosome by a site-directed nuclease, selecting a chromosome with a rearrangement. In some embodiments, the method can utilize a site-directed nuclease is selected from the group consisting of meganucleases (MNs), zinc-finger nucleases (ZFNs), transcription-activator like effector nucleases (TALENs), Cas9 nuclease, Cfp1 nuclease, dCas9-FokI, dCpf1-FokI, chimeric Cas9/Cpf1-cytidine deaminase, chimeric Cas9/Cpf1-adenine deaminase, chimeric FEN1-FokI, and Mega-TALs, a nickase Cas9 (nCas9), chimeric dCas9 non-FokI nuclease and dCpf1 non-FokI nuclease. In some embodiment of the method, the chromosome rearrangement comprises a deletion, duplication, inversion, or translocation. In some embodiments of the method, the chromosome rearrangement causes a modification of gene expression. In some embodiments of the method, the gene expression modification includes regulation at precursor mRNA level, or at mature mRNA level or at translation level. In some embodiments of the method, the chromosome rearrangement includes chromosomes from two species when the chromosomes can be grouped in one nuclei such as in an interspecific hybrid. In some embodiments of the method, the chromosome rearrangement leads to new allele generation via fusing at least two alleles or two components from different alleles. In some embodiments of the method, chromosome rearrangement is targeted to a promoter, exon, intron, or transcription terminator. In some embodiments of the method, chromosome rearrangement causes a modification of gene expression of different genes with sequence similarity to the rearranged gene. In further embodiments of the method, the deletion, duplication, inversion, or translocation is no less than 19 base pairs.

The present invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the present invention.

### EXAMPLES

### Example 1: Gene Inversion by Genome Editing

To test gene inversion by genome editing, a target was identified in the genome of rice (Oryza sativa). The target gene is DENSE AND ERECT PANICLE 1 (DEP1, SEQ ID NO: 1). The Japonica rice dep1 mutant contains a 625 bp deletion close to the 3′end of DEP1. The mutant has dense and erect panicles with a higher grain number and lower plant height than wild type (Huang et al., 2009, Nat Genet 41: 494-497. Indica rice has a wild type copy of the DEP1 gene. For the examples described here, the DEP1 was targeted for gene inversion by genome editing.

Binary vector 22603 comprised an expression cassette (SEQ ID NO: 2) which produced a guide RNA-B (gRNA-B, gtccaagctgcggatgcaa, SEQ ID NO: 3) targeting exon 5 of DEP1 and a second expression cassette with gRNA-D also targeting exon 5 (gtgccctgaatgttcctgt, SEQ ID NO: 4). Binary vector 22604 comprised an expression cassette (SEQ ID NO: 5) which produced a guide RNA-A (actgcagtgcgtgctgcgc, SEQ ID NO: 6) and a second expression cassette with gRNA-D, a third expression cassette which produced gRNA-B and a fourth expression cassette which produced a guide RNA-C(cccaatgcaaacccgattg, SEQ ID NO: 7). All expression cassettes in each binary vector are part of a single transgene.

All binary vectors described here comprise an expression cassette to express a Cas9 endonuclease (WO16106121, incorporated by reference in its entirety herein) and a second expression cassette to express a selectable marker for transformation.

The rice (Oryza sativa) inbred line IR58025B was used for the Agrobacterium-mediated transformation experiments essentially following the protocols for transformation, selection, and regeneration as described in Gui et al. 2014 (Plant Cell Rep 33: 1081-1090, herein incorporated by reference). The transgenic rice lines were grown in a greenhouse with 16 h light/30° C. and 8 h dark/22° C.

Leaf tissue from T0 transgenic events were sampled and used for genomic DNA extraction followed by TaqMan analysis. TaqMan analysis was essentially carried out as described in Ingham et al. (Biotechniques 31(1):132-4, 136-40, 2001), herein incorporated by reference. TaqMan was performed to detect the existence of the Cas9 gene (Table 1, SEQ ID NOs: 9-10 are the primers; SEQ ID NO: 11 is the probe); and a serial of Taqman assays targeted mutations in DEP1 (SEQ ID NOs: 12-20). To detect mutations in DEP1, the forward primer and the reverse primer flank the protospacer target sequence and the probe hybridizes to a region of the protospacer which includes the Cas9 cutting site and the PAM. If a mutation (typically an indel) is introduced at the Cas9 cutting site, the probe will not bind to the target sequence, and therefore will not generate fluorescence (signal 0). The characterization of the genotypes are based on the TaqMan analysis of DEP1 (Table 2).

TABLE 1
SEQ ID NOs of Taqman assays

| SEQ | | | |
|---|---|---|---|
| ID | Oligo | | |
| NO | Type: | Nucleotide Sequence | Target |
| 9 | Forward | TTGTGCTGCTCCACGAACA | Cas9 |
| | primer | | |
| 10 | Reverse | GCCAGCCACTACGAGAAGCT | |
| | primer | | |
| 11 | Probe | CTGCTTCTGCTCGTTGTCCTCCGG | |
| 12 | Forward | TGCGACGAGCCATGCTG | gRNA-A |
| | primer | | |
| 13 | Reverse | GCAGTCTGGACTACAGCATGACC | |
| | primer | | |
| 14 | Probe | CAGCGCAGCACGC | |
| 15 | Forward | CTTGCGCCCAATGCAAAC | gRNA-C |
| | primer | | |
| 16 | Reverse | GCAGCTACAGCAATTGGTAGAGC | |
| | primer | | |
| 17 | Probe | ACGAGCCACAATCG | |
| 18 | Forward | CCTCCCGAAACCGTCGT | gRNA-D |
| | primer | | |
| 19 | Reverse | CGACAACCCTCTGTACAATTCTTG | |
| | primer | | |
| 20 | Probe | CACACCCACAGGAACA | |

TABLE 2
Genotype of T0 edits based on Taqman analysis

| | | T0 editing | | | | |
|---|---|---|---|---|---|---|
| Construct | T0 event ID | pattern | Cas9 | gRNA-A | gRNA-C | gRNA-D |
| 22603 | RIET142202A130A | Homozygous | >2 | 2 | 1 | 0 |
| | | deletion | | | | |
| 22603 | RIET142202A049A | Heterozygous | >2 | 2 | 2 | 0 |
| | | inversion | | | | |
| 22604 | RIET142300A014A | Heterozygous | >2 | 0 | 0 | 0 |
| | | deletion | | | | |
| 22604 | RIET142500B024A | Heterozygous | >2 | 1 | 1 | 1 |
| | | inversion | | | | |

Leaf tissue from T0 events was sampled and used for genomic DNA extraction. The DEP1 gene fragment was amplified by PCR using primers 5′-AAAGACCAAGGTGCCTCA-3′ (SEQ ID NO: 21) and 5′-TGGTTCAACCTCGTCTCATA-3′(SEQ ID NO: 22). The PCR products were isolated by gel electrophoresis with target size, and cloned into the pCR-Blunt vector (Invitrogen). 15-30 colonies per amplicon were sequenced using M13 forward and reverse primers located in the pCR-Blunt vector using the Sanger sequencing method. Sequences were assembled and analysed by alignment to the wild-type DEP1 sequence using both Vector-NTI Advance 11 (Invitrogen) and BLAST analysis.

RIET142202A049A and RIET142500B024A were two edits with inversions in exon5 (FIG. 1). In RIET142202A049A, a 413 bp fragment was inverted between gRNA-B and gRNA-D (genomic sequence, SEQ ID NO: 23). In following expression study, RIET142202A130A from same construct 22603 was selected as control, with a 444 bp deletion between gRNA-B and gRNA-D (genomic sequence, SEQ ID NO: 24). Within edits from 22604, RIET142500B024A was identified with an inversion between gRNA-A and gRNA-B (genomic sequence, SEQ ID NO: 25). Similarly RIET142300A014A was selected as expression control in further (genomic sequence, SEQ ID NO: 26).

### Example 2: Combine Mutated DEP1 with Wild-Type DEP1

To check expression of wild-type DEP1 when inverted DEP1 existences, a workflow was designed as FIG. 2. T1 seeds were harvested from selfed T0 plants, then sowed into germination tray for 2 weeks in greenhouse with 16 h light/30° C. and 8 h dark/22° C. Leaf tissue from T1 plants was sampled and used for genomic DNA extraction. The genotyping primers for 14SBC500773 (T1 seeds of RIET142202A130A) were 5′-TCTTTGCTGCTGTTGCAAGT-3′ (sense primer, SEQ ID NO: 27) and 5′-TCAACCACTGAGACAGCATGG-3′ (antisense primer, SEQ ID NO: 28). The PCR products were isolated by gel electrophoresis (FIG. 3). Similar process was applied to genotype other events.

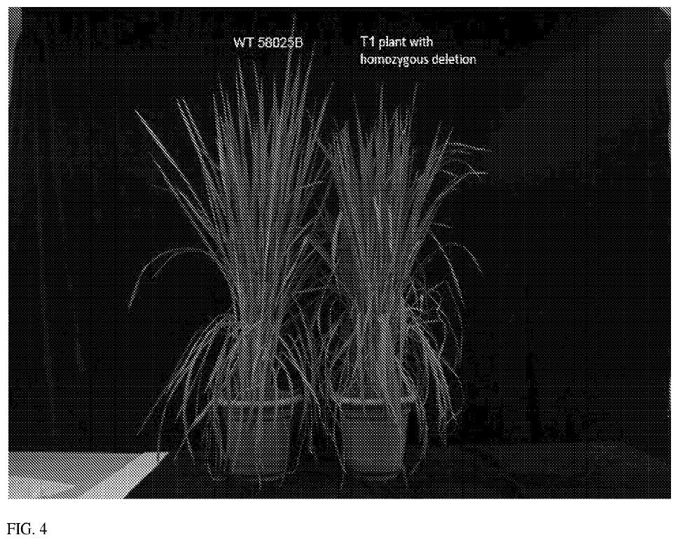

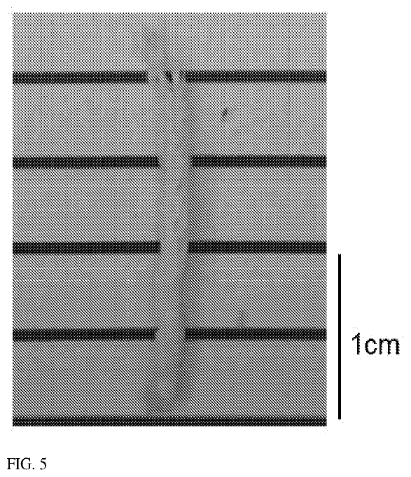

Selected T1 plants were transferred to big pots in greenhouse under same condition (FIG. 4). Meanwhile 58025 wild-type seeds were sowed and transferred to big pots in parallel. At flowering stage, pollen of 58025B wild-type plants were collected and fertilized to homozygous-delated and inverted plants to generate F1 seeds (Table 3).

TABLE 3
Pedigree of F1 seeds

| | | | | F1 plant |
|---|---|---|---|---|
| Construct | T0 event ID | T1 seed ID | F1 seed ID | genotype |
| 22603 | RIET142202A130A | 14SBC500773 | 17SBC500140 | WT/Deletion |
| 22603 | RIET142202A049A | 14SBC500839 | 17SBC500143 | WT/Inversion |
| 22604 | RIET142300A014A | 14SBC500776 | 17SBC500146 | WT/Deletion |
| 22604 | RIET142500B024A | 14SBC500929 | 17SBC500149 | WT/Inversion |

### Example 3: Compare Expression of Wild-Type OsDEP1

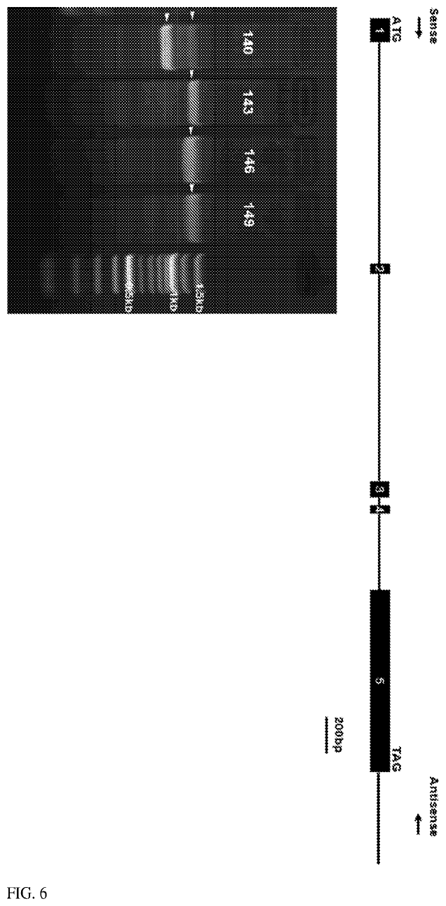

2-3 cm young panicles were sampled from F1 plants at early booting stage (FIG. 5); 5-6 young panicles were sampled from each genotype, RNA isolation and cDNA synthesis were handled sample by sample. RNA was isolated following standard protocol via Invitrogen TRIzol™, and cDNA was synthesized via Superscript™ III first-strand synthesis system (Invitrogen). Firstly, we confirmed the two DEP1 alleles were transcribed in F1 panicles. Sense primer 5′-CTGGAGGTGCAGATCCTGAG-3′ (sense primer, locating in exon 1, SEQ ID NO: 29) and 5′-CTTCAATGGTTCAACCTCGTC-3′ (antisense primer, locating in 3′ UTR, SEQ ID NO: 30) were used (FIG. 6). With the primer pair, amplicon from wild-type 58025B was 1467 bp in size; in F1 plants of 17SBC500140, there were two bands, one for wild-type DEP1, one for DEP1 with 444 bp deletion. In 17SBC500146 and 17SBC500149, the amplicons were similar in size from two alleles, with a 102 bp deletion compared to wild type DEP1 amplicon. Further the existence of DEP1 transcripts with deletion and inversion was confirmed by colony sequencing.

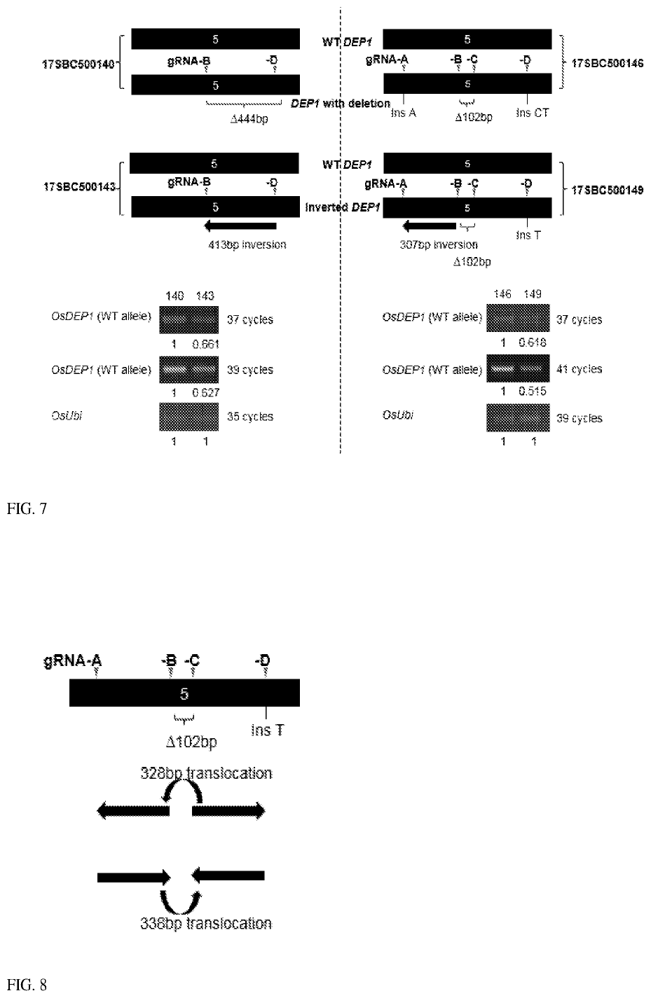

Then mixed the cDNA from each sample in same genotype, and compared wild-type DEP1 expression between WT/deletion and WT/inversion via semi qRT-PCR. Rice ubiquitin (OS03g0234200) was selected for expression control with primers 5′-CCAGCAGCGGCTGATCTTC-3′ (SEQ ID NO: 31) and 5′-CAGGCGCGCATAGCATGAGAA-3′ (SEQ ID NO: 32). Wild-type DEP1 specific primer set, 5′-ATGGGCTGCCACCATGGATAA-3′ (SEQ ID NO: 33) and 5′-CAGCTTGGAAGGCCACAG-3′ (SEQ ID NO: 34), was designed to amplify. PCR products were isolated via gel electrophoresis and quantified by AlphaImager HP software based on area size. Expression ratio was calculated by area of wild-type DEP1 bands in F1 with inversion divided by that in F1 with deletion, then adjusted by ratio of expression of ubiquitin controls (FIG. 7, Table 4). Both types of inversions was able to reduce expression of wild-type DEP1.

TABLE 4

| | 17SBC500140 | 17SBC500143 | | 17SBC500140 | 17SBC500143 |
|---|---|---|---|---|---|
| OsDEP1(WT allele) | 1 | 0.661 | OsDEP1(WT allele) | 1 | 0.618 |
| OsDEP1(WT allele) | 1 | 0.627 | OsDEP1(WT allele) | 1 | 0.515 |
| OsUbi | 1 | 1 | OsUbi | 1 | 1 |

### Example 4: Translocation Via Genome Editing

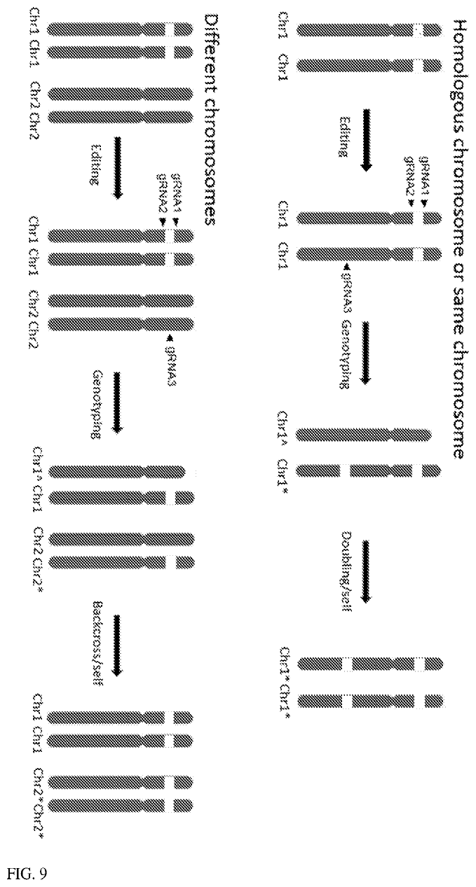

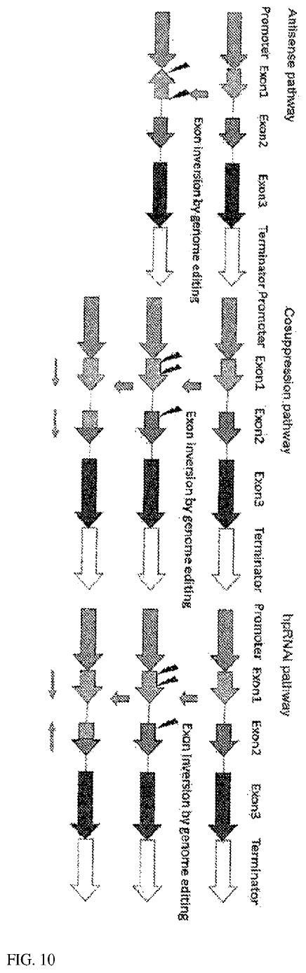

Same process as indicated in Example 1, an E0 plant, RIET142500A084A, was identified with two translocations (FIG. 8, SEQ ID NO: 105, SEQ ID NO: 35). There were two fragments freed from gRNA-A & -B region and gRNA-C & -D region, then instead of generating inversions in same position. Part of gene or the whole gene (including the promoter and terminator region) or multiple genes could be translocated to a new position nearby or in another chromosome. Via targeting the whole genes or multiple genes translocate, duplication can be achieved based on the designs in FIG. 9. Then expression of gene (s) can be up-regulated with more copies. In another side, if genomes form two species grouped in one nuclei, allele exchange can be achieved via genome editing. Duplication with too many copies can also lead to gene silencing; like HA412, a high-oleic sunflower inbred, has 3 intact copies of HaFAD2-1, but with no expression.

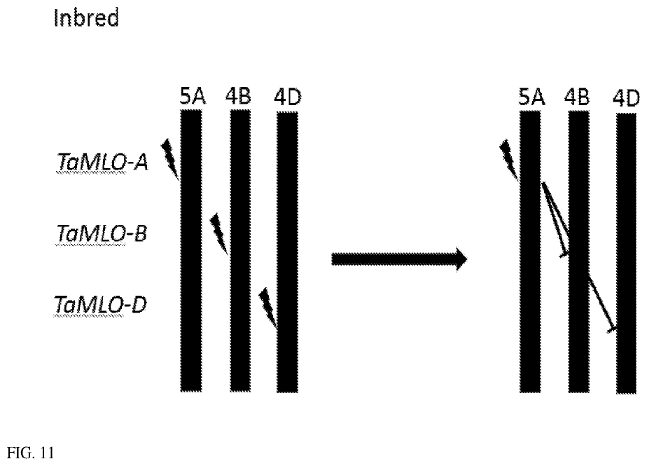

Part of gene can be translocated in same region or new region (s). For translocations in same region, a partial duplication or a hairpin-loop structure can be generated (FIG. 10). In sunflower, partial duplication of HaFAD2-1 (ODS) silences intact ODS gene and leads to high-oleic in kernels (Mol Genet Genomics (2009) 281:43-54); which also can silence other wild-type HaFAD2-1 in hybrid generated. Similar design can provide a non-transgenic gene silencing tool. Like TaMLO, edited TaMLO-A can silence expression of B and D alleles which can achieve disease resistance but with alleviated growth penalty in triple-mutate (FIG. 11). Paralogs could be also modified in expression using same strategy. Translocation can also fuse genes and generate a new gene or a new allele of same gene.

What is claimed is:

1) A method of reducing expression a target gene comprised of:
a) Introducing into a cell a nuclease capable of site-directed DNA cleavage at a target genomic site;
b) Making two or more double strand cuts within a single target gene;
c) Selecting for a cell where the double strand cuts have been repaired with the intervening DNA inverted;
d) reducing expression of the target gene.

2) The method of claim 1, wherein the nuclease is selected from the group consisting of meganucleases (MNs), zinc-finger nucleases (ZFNs), transcription-activator like effector nucleases (TALENs), Cas9 nuclease, Cfp1 nuclease, dCas9-FokI, dCpf1-FokI, chimeric Cas9/Cpf1-cytidine deaminase, chimeric Cas9/Cpf1-adenine deaminase, chimeric FEN1-FokI, and Mega-TALs, a nickase Cas9 (nCas9), chimeric dCas9 non-FokI nuclease and dCpf1 non-FokI nuclease.

3) The method of claim 1 wherein the double strand cuts in the target gene are located in the promoter, UTR, exon, intron, or gene-gene junction region.

4) The method of claim 1 wherein the cell of claim 1 has a haploid, diploid, polyploid, or hexiploid genome.

5) The method of claim 1 wherein the target gene is recessive or semi-dominant.

6) The method of claim 1 further comprising one or more guide sequences.

7) The method of claim 6 wherein the one or more guide sequences comprise two or more guide sequences.

8) The method of claim 1 wherein the cell is a plant cell.

9) A method of rearranging a chromosome by genome editing, comprising:
a. generating at least one breakage in the chromosome by a site-directed nuclease;
b. selecting a chromosome with a rearrangement.

10) The method of claim 9, wherein the site-directed nuclease is selected from the group consisting of meganucleases (MNs), zinc-finger nucleases (ZFNs), transcription-activator like effector nucleases (TALENs), Cas9 nuclease, Cfp1 nuclease, dCas9-FokI, dCpf1-FokI, chimeric Cas9/Cpf1-cytidine deaminase, chimeric Cas9/Cpf1-adenine deaminase, chimeric FEN1-FokI, and Mega-TALs, a nickase Cas9 (nCas9), chimeric dCas9 non-FokI nuclease and dCpf1 non-FokI nuclease.

11) The method of claim 9, wherein the chromosome rearrangement comprises a deletion, duplication, inversion, or translocation.

12) The method of claim 9, wherein the chromosome rearrangement causes a modification of gene expression.

13) The method of claim 9, wherein the gene expression modification includes regulation at precursor mRNA level, or at mature mRNA level or at translation level.

14) The method of claim 9, wherein the chromosome rearrangement includes chromosomes from two species when the chromosomes can be grouped in one nuclei such as in an interspecific hybrid.

15) The method of claim 9, wherein the chromosome rearrangement leads to new allele generation via fusing at least two alleles or two components from different alleles.

16) The method of claim 11, wherein chromosome rearrangement is targeted to a promoter, exon, intron, or transcription terminator.

17) The method of claim 12, chromosome rearrangement causes a modification of gene expression of different genes with sequence similarity to the rearranged gene.

18) The method of claim 11, wherein the deletion, duplication, inversion, or translocation is no less than 19 base pairs.


SEQUENCE LISTING (35 sequences)

SEQ ID NO: 1 - DNA, 4174 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggtgagcgccccgcggcggcggcggctgcgtttttctctataggtttctctttcacactcgctcgctcgaaattctcggggcccgagctctacttgcttcgtcttcctttgactttaccgattaattttaaaaaaaaggagatccgattcgccgcgcatttttcaaaacccaagcggccgagtacggagctacccgctactgcaagtaggatgctgtgaagtgtacagtaatggcgttgttaattgcggtagctagtgctattctagtacttgtagtactgtttctaggcggaggtgaatcacggcgccatcaatccgaggctggcgagacaagcttggccctctttgggcgtggcgccatggctgtactacctttgtcgttgtttggttgggctcctcgttggagaaaagaagagcgtgggcatggacaactgacctgagtggccttgtcagggagagccatagcagtggacgtgtctatctccgccattgcttcgtcgacactggacgtgcagacggcatggccatgagggctttgcacgatgggtggtgccgtgttggtgttatgggctgccaccatggtttgaggcttttgatgttgctagattttgtgtttaacgagggagggaagaatgtgttgttcttgacactgtgctgtgcttttaaggagcagagatttcagaagctcttcagatatcagagaacttctttgtagtagtaatcaaatgcgctttagacatctttttatcgtttcttgcaaggtcagtccctgctttggtacccgatctcgcttttgtgcaacatcaaagttacacttacacagtaaagcaggaatctttatgggaccgttcgtactggtcaattactccaggctttgattaatgggttttaagttttaaccgcagatttggtacaagtaacaacctttatttactttttatttctgcaactgtgtcttttaacatgaaagaatccagctccattcaaaagtttagtttttattttccattgtggtgcatggtcactcagcctgcagtactgaattatcaaaattttcttttgtcatttctctcatgttaagtgcatagtctattttacttcaacaggtagaaaaacttttgtgggtttgtttctagctcaaggaggaaattcatgggtttgcatctagcacatgagagaataatattggtctaacacaaagctccttttgtaggatgagcttcacttccttgaaggagctcagcccgtttctcgttctggatgcattaaagagtatgtactactgcccttcatgcattacagatattttgtttttaagtttttagaaatttgaagagcttatgtcaagtatgaaatgtcagcttaattttattgctgtccttatctaatgtcttatgctctgttttataaaatttggttgcattttctcccccagggaaaaatcttgtataagtgtgttatgtacttatgtgtataaaatcttgttgcacttgtatgtcacacttaggccctgtttagatcctccaaaatggcaaaagttttgccattttgaagcaccttttgccattttggatctaaacactagtaacaaaacttgacaatttggcatttggcatttgctagtctatagtagcaaattgtgccaaaaagtgctttggaaccactccctctttctttctctctctcactttagtgctagaatggcaaaagtttaggatgcatctaaacaccaactagtacttttacaataccaaaacttttgccatttgccatttgctatttcaaatggatataaacagggccttagcaaatcaccatatgttaaaattaccttgggatgaaaaagaaaaaggaaaccagcattgaagtcttgtttgaaatgcatatgtacttgtaccattacagaaattcttaaaactgctgtcttgacagctacttatcaaacagccccacctgcatcataacgttcctagtggtgcctataactctgcctcagttattattttgtggcccactggtccaacaatttgaaaaaaattatattgaacagtagtatgacgtcctctttgcttaagttccatattacagctcatagtcctgagatttgtttcaccgattctttccatgcgatgtgcacatattcttattcaatttaaaaaatgaaagcagattatttttaacaagtaacctatcacgttagcttaacattgtatatttgtggtggaattatgtaatattccgatatcgcatttgaagttttgaacatgtgtgctcaaattgagggacacatgactgtagtgaaagcaaatataaatgtctgaacaatggactatactttgtattcattactacaagttatgtccttttgcaggttgctaatgtcctcttacattacttgtcaggataaatgagtttgttggtacaaaacatgacccactaataccaacgtatggcctctaaactttcagttcccccattttaagcatgttcgctgtttatttacgagttttgacattgttttttccttttccagaaagagaaggaggcacagatcttgccgtctttttcggtggatcgggtatgttttgatccaatatagtttgctcgcaggttctgaggggcaagaacattcaaatatctataatgttttctgttggattcaacattcatcactatttccctcgaaaaaaaaacattcgtcactattggaattgaaagtctgaaagtgcctctagtccctttgtatgttaaaagtcaataaacaagcagtagttttctatatgccacattaatattattgacgcattttaaaaagcaaactagtccagggatgtaatcatctttgttatctaaaactaaaaaaggaaaaactagtgcttttttacattaacattgatttttttgcggctgaaattacatgtagaaactttggcataataatctgtactactgccaaactgagcttttacatggtgaaaatattttccctgcagatcaaaattgtgtatctgcatttcatgtctttgctgctgttgcaagtgctcacccaagtgcaaaagaccaaggtgcctcaattgttcttgcagctcatgctgcgacgagccatgctgtaagccaaactgcagtgcgtgctgcgctgggtcatgctgtagtccagactgctgctcatgctgtaaacctaactgcagttgctgcaagaccccttcttgctgcaaaccgaactgctcgtgctcctgtccaagctgcagctcatgctgcgatacatcgtgctgcaaaccgagctgcacctgcttcaacatcttttcatgcttcaaatccctgtacagctgcttcaagatcccttcatgcttcaagtcccagtgcaactgctctagccccaattgctgcacttgcacccatccaagctgtagctgcaagggctgtgcctgtccaagctgtggatggcaacggctgtggctgtccaagctgcggatgcaacggttgtggctgtccaagctgcggttgcaacggctgtggccttccaagctgcggttgcaacggctgcggctcgtgctcttgcgcccaatgcaaacccgattgtggctcgtgctctaccaattgctgtagctgcaagccaagctgcaacggctgctgcggcgagcagtgctgccgctgcgcggactgcttctcctgctcgtgccctcgtagctccagctgcttcaacatcttcaaatgctcctgcgctggctgctgctcgagcctgtgcaagtgcccctgcacgacgcagtgcttcagctgccagtcgtcatgctgcaagcggcagccttcgtgctgcaagtgccagtcgtcttgctgcgaggggcagccttcctgctgcgagggacactgctgcagcctcccgaaaccgtcgtgccctgaatgttcctgtgggtgtgtctggtcttgcaagaattgtacagagggttgtcgatgcccacggtgtcgtaacccatgctgtctcagtggttgcttatgttgatctagatccttttttggttgtcgtttttcttgtattttttagttgttag

SEQ ID NO: 2 - DNA, 480 bp, Artificial Sequence
  Oryza sativa
gggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagtccaagctgcggatgcaagttttagagctagaaatagcaagttaaaataaggctagtccgttatcaacttgaaaaagtggcaccgagtcggtgcttttttttt

SEQ ID NO: 3 - DNA, 19 bp, Oryza sativa
gtccaagctgcggatgcaa

SEQ ID NO: 4 - DNA, 19 bp, Oryza sativa
gtgccctgaatgttcctgt

SEQ ID NO: 5 - DNA, 2222 bp, Artificial Sequence
  Oryza Sativa
gggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcaactgcagtgcgtgctgcgcgttttagagctagaaatagcaagttaaaataaggctagtccgttatcaacttgaaaaagtggcaccgagtcggtgctttttttttaagctttttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttggtgccctgaatgttcctgtgttttagagctagaaatagcaagttaaaataaggctagtccgttatcaacttgaaaaagtggcaccgagtcggtgctttttttttgggaccggggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcagtccaagctgcggatgcaagttttagagctagaaatagcaagttaaaataaggctagtccgttatcaacttgaaaaagtggcaccgagtcggtgctttttttttgggaccgtttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgcccaatgcaaacccgattggttttagagctagaaatagcaagttaaaataaggctagtccgttatcaacttgaaaaagtggcaccgagtcggtgcttttttttt

SEQ ID NO: 6 - DNA, 19 bp, Oryza sativa
actgcagtgcgtgctgcgc

SEQ ID NO: 7 - DNA, 19 bp, Oryza sativa
cccaatgcaaacccgattg

SEQ ID NO: 8 - DNA, 4962 bp, Artificial Sequence
  Xanthomonas spp, Zea mays
agtcaaagattcaaatagaggacctaacagaactcgccgtaaagactggcgaacagttcatacagagtctcttacgactcaatgacaagaagaaaatcttcgtcaacttggtggagcacgacacgctagtctactccaaaaatatcaaagatacagtctcagaagaccaaagggcaattgagacttttcaacaaagggtaatatccggaaacctcctcggattccattgcccagctatctgtcacttaattgtgaagatagtggaaaaggaaggtggctcctacaaatgccatcattgcgataaaggaaaggccatcgttgaagatgcctctgccgacagtggtcccaaagatggacccccacccacgaggagcatcgtggtaaaagaagacgttccaaccacgtcttcaaagcaagtggattgatgtgatatctccactgacgtaagggatgacgcacaatcccactatccttcgcaagacccttcctctatataaggaagttcatttcatttggagaggataattatccaccatggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggatccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtgaagcttgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 9 - DNA, 19 bp, Artificial Sequence
  Xanthomonas spp
ttgtgctgctccacgaaca

SEQ ID NO: 10 - DNA, 20 bp, Artificial Sequence
  Xanthomonas spp
gccagccactacgagaagct

SEQ ID NO: 11 - DNA, 24 bp, Artificial Sequence
  Xanthomonas spp
ctgcttctgctcgttgtcctccgg

SEQ ID NO: 12 - DNA, 17 bp, Oryza sativa
tgcgacgagccatgctg

SEQ ID NO: 13 - DNA, 23 bp, Oryza sativa
gcagtctggactacagcatgacc

SEQ ID NO: 14 - DNA, 13 bp, Oryza sativa
cagcgcagcacgc

SEQ ID NO: 15 - DNA, 18 bp, Oryza sativa
cttgcgcccaatgcaaac

SEQ ID NO: 16 - DNA, 23 bp, Oryza sativa
gcagctacagcaattggtagagc

SEQ ID NO: 17 - DNA, 14 bp, Oryza sativa
acgagccacaatcg

SEQ ID NO: 18 - DNA, 17 bp, Oryza sativa
cctcccgaaaccgtcgt

SEQ ID NO: 19 - DNA, 24 bp, Oryza sativa
cgacaaccctctgtacaattcttg

SEQ ID NO: 20 - DNA, 16 bp, Oryza sativa
cacacccacaggaaca

SEQ ID NO: 21 - DNA, 18 bp, Oryza sativa
aaagaccaaggtgcctca

SEQ ID NO: 22 - DNA, 20 bp, Oryza sativa
tggttcaacctcgtctcata

SEQ ID NO: 23 - DNA, 4176 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggtgagcgccccgcggcggcggcggctgcgtttttctctataggtttctctttcacactcgctcgctcgaaattctcggggcccgagctctacttgcttcgtcttcctttgactttaccgattaattttaaaaaaaaggagatccgattcgccgcgcatttttcaaaacccaagcggccgagtacggagctacccgctactgcaagtaggatgctgtgaagtgtacagtaatggcgttgttaattgcggtagctagtgctattctagtacttgtagtactgtttctaggcggaggtgaatcacggcgccatcaatccgaggctggcgagacaagcttggccctctttgggcgtggcgccatggctgtactacctttgtcgttgtttggttgggctcctcgttggagaaaagaagagcgtgggcatggacaactgacctgagtggccttgtcagggagagccatagcagtggacgtgtctatctccgccattgcttcgtcgacactggacgtgcagacggcatggccatgagggctttgcacgatgggtggtgccgtgttggtgttatgggctgccaccatggtttgaggcttttgatgttgctagattttgtgtttaacgagggagggaagaatgtgttgttcttgacactgtgctgtgcttttaaggagcagagatttcagaagctcttcagatatcagagaacttctttgtagtagtaatcaaatgcgctttagacatctttttatcgtttcttgcaaggtcagtccctgctttggtacccgatctcgcttttgtgcaacatcaaagttacacttacacagtaaagcaggaatctttatgggaccgttcgtactggtcaattactccaggctttgattaatgggttttaagttttaaccgcagatttggtacaagtaacaacctttatttactttttatttctgcaactgtgtcttttaacatgaaagaatccagctccattcaaaagtttagtttttattttccattgtggtgcatggtcactcagcctgcagtactgaattatcaaaattttcttttgtcatttctctcatgttaagtgcatagtctattttacttcaacaggtagaaaaacttttgtgggtttgtttctagctcaaggaggaaattcatgggtttgcatctagcacatgagagaataatattggtctaacacaaagctccttttgtaggatgagcttcacttccttgaaggagctcagcccgtttctcgttctggatgcattaaagagtatgtactactgcccttcatgcattacagatattttgtttttaagtttttagaaatttgaagagcttatgtcaagtatgaaatgtcagcttaattttattgctgtccttatctaatgtcttatgctctgttttataaaatttggttgcattttctcccccagggaaaaatcttgtataagtgtgttatgtacttatgtgtataaaatcttgttgcacttgtatgtcacacttaggccctgtttagatcctccaaaatggcaaaagttttgccattttgaagcaccttttgccattttggatctaaacactagtaacaaaacttgacaatttggcatttggcatttgctagtctatagtagcaaattgtgccaaaaagtgctttggaaccactccctctttctttctctctctcactttagtgctagaatggcaaaagtttaggatgcatctaaacaccaactagtacttttacaataccaaaacttttgccatttgccatttgctatttcaaatggatataaacagggccttagcaaatcaccatatgttaaaattaccttgggatgaaaaagaaaaaggaaaccagcattgaagtcttgtttgaaatgcatatgtacttgtaccattacagaaattcttaaaactgctgtcttgacagctacttatcaaacagccccacctgcatcataacgttcctagtggtgcctataactctgcctcagttattattttgtggcccactggtccaacaatttgaaaaaaattatattgaacagtagtatgacgtcctctttgcttaagttccatattacagctcatagtcctgagatttgtttcaccgattctttccatgcgatgtgcacatattcttattcaatttaaaaaatgaaagcagattatttttaacaagtaacctatcacgttagcttaacattgtatatttgtggtggaattatgtaatattccgatatcgcatttgaagttttgaacatgtgtgctcaaattgagggacacatgactgtagtgaaagcaaatataaatgtctgaacaatggactatactttgtattcattactacaagttatgtccttttgcaggttgctaatgtcctcttacattacttgtcaggataaatgagtttgttggtacaaaacatgacccactaataccaacgtatggcctctaaactttcagttcccccattttaagcatgttcgctgtttatttacgagttttgacattgttttttccttttccagaaagagaaggaggcacagatcttgccgtctttttcggtggatcgggtatgttttgatccaatatagtttgctcgcaggttctgaggggcaagaacattcaaatatctataatgttttctgttggattcaacattcatcactatttccctcgaaaaaaaaacattcgtcactattggaattgaaagtctgaaagtgcctctagtccctttgtatgttaaaagtcaataaacaagcagtagttttctatatgccacattaatattattgacgcattttaaaaagcaaactagtccagggatgtaatcatctttgttatctaaaactaaaaaaggaaaaactagtgcttttttacattaacattgatttttttgcggctgaaattacatgtagaaactttggcataataatctgtactactgccaaactgagcttttacatggtgaaaatattttccctgcagatcaaaattgtgtatctgcatttcatgtctttgctgctgttgcaagtgctcacccaagtgcaaaagaccaaggtgcctcaattgttcttgcagctcatgctgcgacgagccatgctgtaagccaaactgcagtgcgtgctgcgctgggtcatgctgtagtccagactgctgctcatgctgtaaacctaactgcagttgctgcaagaccccttcttgctgcaaaccgaactgctcgtgctcctgtccaagctgcagctcatgctgcgatacatcgtgctgcaaaccgagctgcacctgcttcaacatcttttcatgcttcaaatccctgtacaactgcttcaagatcccttcatgcttcaagtcccagtgcaactgctctagccccaattgctgcacttgcacccatccaagctgtagctgcaagggctgtgcctgtccaagctgtggatggcaacggctgtggctgtccaagctgcggatgtggaacattcagggcacgacggtttcgggaggctgcagcagtgtccctcgcagcaggaaggctgcccctcgcagcaagacgactggcacttgcagcacgaaggctgccgcttgcagcatgacgactggcagctgaagcactgcgtcgtgcaggggcacttgcacaggctcgagcagcagccagcgcaggagcatttgaagatgttgaagcagctggagctacgagggcacgagcaggagaagcagtccgcgcagcggcagcactgctcgccgcagcagccgttgcagcttggcttgcagctacagcaattggtagagcacgagccacaatcgggtttgcattgggcgcaagagcaccagccgcagccgttgcaaccgcagcttggaaggccacagccgttggcaaccgcagcttggacagccacaaccgttgtgtgggtgtgtctggtcttgcaagaattgtacagagggttgtcgatgcccacggtgtcgtaacccatgctgtctcagtggttgcttatgttgatctagatccttttttggttgtcgtttttcttgtattttttagttgttag

SEQ ID NO: 24 - DNA, 3730 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggtgagcgccccgcggcggcggcggctgcgtttttctctataggtttctctttcacactcgctcgctcgaaattctcggggcccgagctctacttgcttcgtcttcctttgactttaccgattaattttaaaaaaaaggagatccgattcgccgcgcatttttcaaaacccaagcggccgagtacggagctacccgctactgcaagtaggatgctgtgaagtgtacagtaatggcgttgttaattgcggtagctagtgctattctagtacttgtagtactgtttctaggcggaggtgaatcacggcgccatcaatccgaggctggcgagacaagcttggccctctttgggcgtggcgccatggctgtactacctttgtcgttgtttggttgggctcctcgttggagaaaagaagagcgtgggcatggacaactgacctgagtggccttgtcagggagagccatagcagtggacgtgtctatctccgccattgcttcgtcgacactggacgtgcagacggcatggccatgagggctttgcacgatgggtggtgccgtgttggtgttatgggctgccaccatggtttgaggcttttgatgttgctagattttgtgtttaacgagggagggaagaatgtgttgttcttgacactgtgctgtgcttttaaggagcagagatttcagaagctcttcagatatcagagaacttctttgtagtagtaatcaaatgcgctttagacatctttttatcgtttcttgcaaggtcagtccctgctttggtacccgatctcgcttttgtgcaacatcaaagttacacttacacagtaaagcaggaatctttatgggaccgttcgtactggtcaattactccaggctttgattaatgggttttaagttttaaccgcagatttggtacaagtaacaacctttatttactttttatttctgcaactgtgtcttttaacatgaaagaatccagctccattcaaaagtttagtttttattttccattgtggtgcatggtcactcagcctgcagtactgaattatcaaaattttcttttgtcatttctctcatgttaagtgcatagtctattttacttcaacaggtagaaaaacttttgtgggtttgtttctagctcaaggaggaaattcatgggtttgcatctagcacatgagagaataatattggtctaacacaaagctccttttgtaggatgagcttcacttccttgaaggagctcagcccgtttctcgttctggatgcattaaagagtatgtactactgcccttcatgcattacagatattttgtttttaagtttttagaaatttgaagagcttatgtcaagtatgaaatgtcagcttaattttattgctgtccttatctaatgtcttatgctctgttttataaaatttggttgcattttctcccccagggaaaaatcttgtataagtgtgttatgtacttatgtgtataaaatcttgttgcacttgtatgtcacacttaggccctgtttagatcctccaaaatggcaaaagttttgccattttgaagcaccttttgccattttggatctaaacactagtaacaaaacttgacaatttggcatttggcatttgctagtctatagtagcaaattgtgccaaaaagtgctttggaaccactccctctttctttctctctctcactttagtgctagaatggcaaaagtttaggatgcatctaaacaccaactagtacttttacaataccaaaacttttgccatttgccatttgctatttcaaatggatataaacagggccttagcaaatcaccatatgttaaaattaccttgggatgaaaaagaaaaaggaaaccagcattgaagtcttgtttgaaatgcatatgtacttgtaccattacagaaattcttaaaactgctgtcttgacagctacttatcaaacagccccacctgcatcataacgttcctagtggtgcctataactctgcctcagttattattttgtggcccactggtccaacaatttgaaaaaaattatattgaacagtagtatgacgtcctctttgcttaagttccatattacagctcatagtcctgagatttgtttcaccgattctttccatgcgatgtgcacatattcttattcaatttaaaaaatgaaagcagattatttttaacaagtaacctatcacgttagcttaacattgtatatttgtggtggaattatgtaatattccgatatcgcatttgaagttttgaacatgtgtgctcaaattgagggacacatgactgtagtgaaagcaaatataaatgtctgaacaatggactatactttgtattcattactacaagttatgtccttttgcaggttgctaatgtcctcttacattacttgtcaggataaatgagtttgttggtacaaaacatgacccactaataccaacgtatggcctctaaactttcagttcccccattttaagcatgttcgctgtttatttacgagttttgacattgttttttccttttccagaaagagaaggaggcacagatcttgccgtctttttcggtggatcgggtatgttttgatccaatatagtttgctcgcaggttctgaggggcaagaacattcaaatatctataatgttttctgttggattcaacattcatcactatttccctcgaaaaaaaaacattcgtcactattggaattgaaagtctgaaagtgcctctagtccctttgtatgttaaaagtcaataaacaagcagtagttttctatatgccacattaatattattgacgcattttaaaaagcaaactagtccagggatgtaatcatctttgttatctaaaactaaaaaaggaaaaactagtgcttttttacattaacattgatttttttgcggctgaaattacatgtagaaactttggcataataatctgtactactgccaaactgagcttttacatggtgaaaatattttccctgcagatcaaaattgtgtatctgcatttcatgtctttgctgctgttgcaagtgctcacccaagtgcaaaagaccaaggtgcctcaattgttcttgcagctcatgctgcgacgagccatgctgtaagccaaactgcagtgcgtgctgcgctgggtcatgctgtagtccagactgctgctcatgctgtaaacctaactgcagttgctgcaagaccccttcttgctgcaaaccgaactgctcgtgctcctgtccaagctgcagctcatgctgcgatacatcgtgctgcaaaccgagctgcacctgcttcaacatcttttcatgcttcaaatccctgtacaactgcttcaagatcccttcatgcttcaagtcccagtgcaactgctctagccccaattgctgcacttgcacccatccaagctgtagctgcaagggctgtgcctgtccaagctgtggatggcaacggctgtggctgtccaagctgtgtctggtcttgcaagaattgtacagagggttgtcgatgcccacggtgtcgtaacccatgctgtctcagtggttgcttatgttgatctagatccttttttggttgtcgtttttcttgtattttttagttgttag

SEQ ID NO: 25 - DNA, 4522 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggtgagcgccccgcggcggcggcggctgcgtttttctctataggtttctctttcacactcgctcgctcgaaattctcggggcccgagctctacttgcttcgtcttcctttgactttaccgattaattttaaaaaaaaggagatccgattcgccgcgcatttttcaaaacccaagcggccgagtacggagctacccgctactgcaagtaggatgctgtgaagtgtacagtaatggcgttgttaattgcggtagctagtgctattctagtacttgtagtactgtttctaggcggaggtgaatcacggcgccatcaatccgaggctggcgagacaagcttggccctctttgggcgtggcgccatggctgtactacctttgtcgttgtttggttgggctcctcgttggagaaaagaagagcgtgggcatggacaactgacctgagtggccttgtcagggagagccatagcagtggacgtgtctatctccgccattgcttcgtcgacactggacgtgcagacggcatggccatgagggctttgcacgatgggtggtgccgtgttggtgttatgggctgccaccatggtttgaggcttttgatgttgctagattttgtgtttaacgagggagggaagaatgtgttgttcttgacactgtgctgtgcttttaaggagcagagatttcagaagctcttcagatatcagagaacttctttgtagtagtaatcaaatgcgctttagacatctttttatcgtttcttgcaaggtcagtccctgctttggtacccgatctcgcttttgtgcaacatcaaagttacacttacacagtaaagcaggaatctttatgggaccgttcgtactggtcaattactccaggctttgattaatgggttttaagttttaaccgcagatttggtacaagtaacaacctttatttactttttatttctgcaactgtgtcttttaacatgaaagaatccagctccattcaaaagtttagtttttattttccattgtggtgcatggtcactcagcctgcagtactgaattatcaaaattttcttttgtcatttctctcatgttaagtgcatagtctattttacttcaacaggtagaaaaacttttgtgggtttgtttctagctcaaggaggaaattcatgggtttgcatctagcacatgagagaataatattggtctaacacaaagctccttttgtaggatgagcttcacttccttgaaggagctcagcccgtttctcgttctggatgcattaaagagtatgtactactgcccttcatgcattacagatattttgtttttaagtttttagaaatttgaagagcttatgtcaagtatgaaatgtcagcttaattttattgctgtccttatctaatgtcttatgctctgttttataaaatttggttgcattttctcccccagggaaaaatcttgtataagtgtgttatgtacttatgtgtataaaatcttgttgcacttgtatgtcacacttaggccctgtttagatcctccaaaatggcaaaagttttgccattttgaagcaccttttgccattttggatctaaacactagtaacaaaacttgacaatttggcatttggcatttgctagtctatagtagcaaattgtgccaaaaagtgctttggaaccactccctctttctttctctctctcactttagtgctagaatggcaaaagtttaggatgcatctaaacaccaactagtacttttacaataccaaaacttttgccatttgccatttgctatttcaaatggatataaacagggccttagcaaatcaccatatgttaaaattaccttgggatgaaaaagaaaaaggaaaccagcattgaagtcttgtttgaaatgcatatgtacttgtaccattacagaaattcttaaaactgctgtcttgacagctacttatcaaacagccccacctgcatcataacgttcctagtggtgcctataactctgcctcagttattattttgtggcccactggtccaacaatttgaaaaaaattatattgaacagtagtatgacgtcctctttgcttaagttccatattacagctcatagtcctgagatttgtttcaccgattctttccatgcgatgtgcacatattcttattcaatttaaaaaatgaaagcagattatttttaacaagtaacctatcacgttagcttaacattgtatatttgtggtggaattatgtaatattccgatatcgcatttgaagttttgaacatgtgtgctcaaattgagggacacatgactgtagtgaaagcaaatataaatgtctgaacaatggactatactttgtattcattactacaagttatgtccttttgcaggttgctaatgtcctcttacattacttgtcaggataaatgagtttgttggtacaaaacatgacccactaataccaacgtatggcctctaaactttcagttcccccattttaagcatgttcgctgtttatttacgagttttgacattgttttttccttttccagaaagagaaggaggcacagatcttgccgtctttttcggtggatcgggtatgttttgatccaatatagtttgctcgcaggttctgaggggcaagaacattcaaatatctataatgttttctgttggattcaacattcatcactatttccctcgaaaaaaaaacattcgtcactattggaattgaaagtctgaaagtgcctctagtccctttgtatgttaaaagtcaataaacaagcagtagttttctatatgccacattaatattattgacgcattttaaaaagcaaactagtccagggatgtaatcatctttgttatctaaaactaaaaaaggaaaaactagtgcttttttacattaacattgatttttttgcggctgaaattacatgtagaaactttggcataataatctgtactactgccaaactgagcttttacatggtgaaaatattttccctgcagatcaaaattgtgtatctgcatttcatgtctttgctgctgttgcaagtgctcacccaagtgcaaaagaccaaggtgcctcaattgttcttgcagctcatgctgcgacgagccatgctgtaagccaaactgcagtgcgtgctgcatccacagcttggacaggcacagcccttgcagctacagcttggatgggtgcaagtgcagcaattggggctagagcagttgcactgggacttgaagcatgaagggatcttgaagcagttgtacagggatttgaagcatgaaaagatgttgaagcaggtgcagctcggtttgcagcacgatgtatcgcagcatgagctgcagcttggacaggagcacgagcagttcggtttgcagcaagaaggggtcttgcagcaactgcagttaggtttacagcatgagcagcagtctggactacagcatgacccagccaacggctgtggctgtccaagctgcggatgttgtggctcgtgctctaccaattgctgtagctgcaagccaagctgcaacggctgctgcggcgagcagtgctgccgctgcgcggactgcttctcctgctcgtgccctcgtagctccagctgcttcaacatcttcaaatgctcctgcgctggctgctgctcgagcctgtgcaagtgcccctgcacgacgcagtgcttcagctgccagtcgtcatgctgcaagcggcagccttcgtgctgcaagtgccagtcgtcttgctgcgaggggcagccttcctgctgcgagggacactgctgcagcctcccgaaaccgtcgtgccctgaatgttccttgtgggtgtgtctggtcttgcaagaattgtacagagggttgtcgatgcccacggtgtcgtaacccatgctgtctcagtggttgcttatgttgatctagatccttttttggttgtcgtttttcttgtattttttagttgttaggcctttgattaagttcgaactttcataaatatatggtgtttatcctgtaaagaaatgatgatttcaaggatttttcatagctatgagacgaggttgaaccattgaagagccaatagaccgtgaagatatggagccatacacgctgaataatttttctgacagggtgaggtcgaattggctgccgtttcgcgaggacttttgctgcgcgacggcgaggacagccggggtcccacctgtcagccagctttgcgagctcaatgcttttatttccgccgttgcttcaccacgccgcccggcaaattcggcccgtcaacgacggtaggcctatatctctaatattgaaatccatgcaaattatcgaacgattagcggtgccacgtgacatggcgtggttcagcactcccacacttaaaaaggactaaaatataaaaaacaattttacgttggagta

SEQ ID NO: 26 - DNA, 4073 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggtgagcgccccgcggcggcggcggctgcgtttttctctataggtttctctttcacactcgctcgctcgaaattctcggggcccgagctctacttgcttcgtcttcctttgactttaccgattaattttaaaaaaaaggagatccgattcgccgcgcatttttcaaaacccaagcggccgagtacggagctacccgctactgcaagtaggatgctgtgaagtgtacagtaatggcgttgttaattgcggtagctagtgctattctagtacttgtagtactgtttctaggcggaggtgaatcacggcgccatcaatccgaggctggcgagacaagcttggccctctttgggcgtggcgccatggctgtactacctttgtcgttgtttggttgggctcctcgttggagaaaagaagagcgtgggcatggacaactgacctgagtggccttgtcagggagagccatagcagtggacgtgtctatctccgccattgcttcgtcgacactggacgtgcagacggcatggccatgagggctttgcacgatgggtggtgccgtgttggtgttatgggctgccaccatggtttgaggcttttgatgttgctagattttgtgtttaacgagggagggaagaatgtgttgttcttgacactgtgctgtgcttttaaggagcagagatttcagaagctcttcagatatcagagaacttctttgtagtagtaatcaaatgcgctttagacatctttttatcgtttcttgcaaggtcagtccctgctttggtacccgatctcgcttttgtgcaacatcaaagttacacttacacagtaaagcaggaatctttatgggaccgttcgtactggtcaattactccaggctttgattaatgggttttaagttttaaccgcagatttggtacaagtaacaacctttatttactttttatttctgcaactgtgtcttttaacatgaaagaatccagctccattcaaaagtttagtttttattttccattgtggtgcatggtcactcagcctgcagtactgaattatcaaaattttcttttgtcatttctctcatgttaagtgcatagtctattttacttcaacaggtagaaaaacttttgtgggtttgtttctagctcaaggaggaaattcatgggtttgcatctagcacatgagagaataatattggtctaacacaaagctccttttgtaggatgagcttcacttccttgaaggagctcagcccgtttctcgttctggatgcattaaagagtatgtactactgcccttcatgcattacagatattttgtttttaagtttttagaaatttgaagagcttatgtcaagtatgaaatgtcagcttaattttattgctgtccttatctaatgtcttatgctctgttttataaaatttggttgcattttctcccccagggaaaaatcttgtataagtgtgttatgtacttatgtgtataaaatcttgttgcacttgtatgtcacacttaggccctgtttagatcctccaaaatggcaaaagttttgccattttgaagcaccttttgccattttggatctaaacactagtaacaaaacttgacaatttggcatttggcatttgctagtctatagtagcaaattgtgccaaaaagtgctttggaaccactccctctttctttctctctctcactttagtgctagaatggcaaaagtttaggatgcatctaaacaccaactagtacttttacaataccaaaacttttgccatttgccatttgctatttcaaatggatataaacagggccttagcaaatcaccatatgttaaaattaccttgggatgaaaaagaaaaaggaaaccagcattgaagtcttgtttgaaatgcatatgtacttgtaccattacagaaattcttaaaactgctgtcttgacagctacttatcaaacagccccacctgcatcataacgttcctagtggtgcctataactctgcctcagttattattttgtggcccactggtccaacaatttgaaaaaaattatattgaacagtagtatgacgtcctctttgcttaagttccatattacagctcatagtcctgagatttgtttcaccgattctttccatgcgatgtgcacatattcttattcaatttaaaaaatgaaagcagattatttttaacaagtaacctatcacgttagcttaacattgtatatttgtggtggaattatgtaatattccgatatcgcatttgaagttttgaacatgtgtgctcaaattgagggacacatgactgtagtgaaagcaaatataaatgtctgaacaatggactatactttgtattcattactacaagttatgtccttttgcaggttgctaatgtcctcttacattacttgtcaggataaatgagtttgttggtacaaaacatgacccactaataccaacgtatggcctctaaactttcagttcccccattttaagcatgttcgctgtttatttacgagttttgacattgttttttccttttccagaaagagaaggaggcacagatcttgccgtctttttcggtggatcgggtatgttttgatccaatatagtttgctcgcaggttctgaggggcaagaacattcaaatatctataatgttttctgttggattcaacattcatcactatttccctcgaaaaaaaaacattcgtcactattggaattgaaagtctgaaagtgcctctagtccctttgtatgttaaaagtcaataaacaagcagtagttttctatatgccacattaatattattgacgcattttaaaaagcaaactagtccagggatgtaatcatctttgttatctaaaactaaaaaaggaaaaactagtgcttttttacattaacattgatttttttgcggctgaaattacatgtagaaactttggcataataatctgtactactgccaaactgagcttttacatggtgaaaatattttccctgcagatcaaaattgtgtatctgcatttcatgtctttgctgctgttgcaagtgctcacccaagtgcaaaagaccaaggtgcctcaattgttcttgcagctcatgctgcgacgagccatgctgtaagccaaactgcagtgcgtgctgacgctgggtcatgctgtagtccagactgctgctcatgctgtaaacctaactgcagttgctgcaagaccccttcttgctgcaaaccgaactgctcgtgctcctgtccaagctgcagctcatgctgcgatacatcgtgctgcaaaccgagctgcacctgcttcaacatcttttcatgcttcaaatccctgtacaactgcttcaagatcccttcatgcttcaagtcccagtgcaactgctctagccccaattgctgcacttgcacccatccaagctgtagctgcaagggctgtgcctgtccaagctgtggatcaacggctgtggctgtccaagctgcggatgttgtggctcgtgctctaccaattgctgtagctgcaagccaagctgcaacggctgctgcggcgagcagtgctgccgctgcgcggactgcttctcctgctcgtgccctcgtagctccagctgcttcaacatcttcaaatgctcctgcgctggctgctgctcgagcctgtgcaagtgcccctgcacgacgcagtgcttcagctgccagtcgtcatgctgcaagcggcagccttcgtgctgcaagtgccagtcgtcttgctgcgaggggcagccttcctgctgcgagggacactgctgcagcctcccgaaaccgtcgtgccctgaatgttcccttgtgggtgtgtctggtcttgcaagaattgtacagagggttgtcgatgcccacggtgtcgtaacccatgctgtctcagtggttgcttatgttgatctagatccttttttggttgtcgtttttcttgtattttttagttgttag

SEQ ID NO: 27 - DNA, 20 bp, Oryza sativa
tctttgctgctgttgcaagt

SEQ ID NO: 28 - DNA, 21 bp, Oryza sativa
tcaaccactgagacagcatgg

SEQ ID NO: 29 - DNA, 20 bp, Oryza sativa
ctggaggtgcagatcctgag

SEQ ID NO: 30 - DNA, 21 bp, Oryza sativa
cttcaatggttcaacctcgtc

SEQ ID NO: 31 - DNA, 19 bp, Oryza sativa
ccagcagcggctgatcttc

SEQ ID NO: 32 - DNA, 21 bp, Oryza sativa
caggcgcgcatagcatgagaa

SEQ ID NO: 33 - DNA, 21 bp, Oryza sativa
atgggctgccaccatggataa

SEQ ID NO: 34 - DNA, 18 bp, Oryza sativa
cagcttggaaggccacag

SEQ ID NO: 35 - DNA, 4070 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggtgagcgccccgcggcggcggcggctgcgtttttctctataggtttctctttcacactcgctcgctcgaaattctcggggcccgagctctacttgcttcgtcttcctttgactttaccgattaattttaaaaaaaaggagatccgattcgccgcgcatttttcaaaacccaagcggccgagtacggagctacccgctactgcaagtaggatgctgtgaagtgtacagtaatggcgttgttaattgcggtagctagtgctattctagtacttgtagtactgtttctaggcggaggtgaatcacggcgccatcaatccgaggctggcgagacaagcttggccctctttgggcgtggcgccatggctgtactacctttgtcgttgtttggttgggctcctcgttggagaaaagaagagcgtgggcatggacaactgacctgagtggccttgtcagggagagccatagcagtggacgtgtctatctccgccattgcttcgtcgacactggacgtgcagacggcatggccatgagggctttgcacgatgggtggtgccgtgttggtgttatgggctgccaccatggtttgaggcttttgatgttgctagattttgtgtttaacgagggagggaagaatgtgttgttcttgacactgtgctgtgcttttaaggagcagagatttcagaagctcttcagatatcagagaacttctttgtagtagtaatcaaatgcgctttagacatctttttatcgtttcttgcaaggtcagtccctgctttggtacccgatctcgcttttgtgcaacatcaaagttacacttacacagtaaagcaggaatctttatgggaccgttcgtactggtcaattactccaggctttgattaatgggttttaagttttaaccgcagatttggtacaagtaacaacctttatttactttttatttctgcaactgtgtcttttaacatgaaagaatccagctccattcaaaagtttagtttttattttccattgtggtgcatggtcactcagcctgcagtactgaattatcaaaattttcttttgtcatttctctcatgttaagtgcatagtctattttacttcaacaggtagaaaaacttttgtgggtttgtttctagctcaaggaggaaattcatgggtttgcatctagcacatgagagaataatattggtctaacacaaagctccttttgtaggatgagcttcacttccttgaaggagctcagcccgtttctcgttctggatgcattaaagagtatgtactactgcccttcatgcattacagatattttgtttttaagtttttagaaatttgaagagcttatgtcaagtatgaaatgtcagcttaattttattgctgtccttatctaatgtcttatgctctgttttataaaatttggttgcattttctcccccagggaaaaatcttgtataagtgtgttatgtacttatgtgtataaaatcttgttgcacttgtatgtcacacttaggccctgtttagatcctccaaaatggcaaaagttttgccattttgaagcaccttttgccattttggatctaaacactagtaacaaaacttgacaatttggcatttggcatttgctagtctatagtagcaaattgtgccaaaaagtgctttggaaccactccctctttctttctctctctcactttagtgctagaatggcaaaagtttaggatgcatctaaacaccaactagtacttttacaataccaaaacttttgccatttgccatttgctatttcaaatggatataaacagggccttagcaaatcaccatatgttaaaattaccttgggatgaaaaagaaaaaggaaaccagcattgaagtcttgtttgaaatgcatatgtacttgtaccattacagaaattcttaaaactgctgtcttgacagctacttatcaaacagccccacctgcatcataacgttcctagtggtgcctataactctgcctcagttattattttgtggcccactggtccaacaatttgaaaaaaattatattgaacagtagtatgacgtcctctttgcttaagttccatattacagctcatagtcctgagatttgtttcaccgattctttccatgcgatgtgcacatattcttattcaatttaaaaaatgaaagcagattatttttaacaagtaacctatcacgttagcttaacattgtatatttgtggtggaattatgtaatattccgatatcgcatttgaagttttgaacatgtgtgctcaaattgagggacacatgactgtagtgaaagcaaatataaatgtctgaacaatggactatactttgtattcattactacaagttatgtccttttgcaggttgctaatgtcctcttacattacttgtcaggataaatgagtttgttggtacaaaacatgacccactaataccaacgtatggcctctaaactttcagttcccccattttaagcatgttcgctgtttatttacgagttttgacattgttttttccttttccagaaagagaaggaggcacagatcttgccgtctttttcggtggatcgggtatgttttgatccaatatagtttgctcgcaggttctgaggggcaagaacattcaaatatctataatgttttctgttggattcaacattcatcactatttccctcgaaaaaaaaacattcgtcactattggaattgaaagtctgaaagtgcctctagtccctttgtatgttaaaagtcaataaacaagcagtagttttctatatgccacattaatattattgacgcattttaaaaagcaaactagtccagggatgtaatcatctttgttatctaaaactaaaaaaggaaaaactagtgcttttttacattaacattgatttttttgcggctgaaattacatgtagaaactttggcataataatctgtactactgccaaactgagcttttacatggtgaaaatattttccctgcagatcaaaattgtgtatctgcatttcatgtctttgctgctgttgcaagtgctcacccaagtgcaaaagaccaaggtgcctcaattgttcttgcagctcatgctgcgacgagccatgctgtaagccaaactgcagtgcgtgctgggaacattcagggcacgacggtttcgggaggctgcagcagtgtccctcgcagcaggaaggctgcccctcgcagcaagacgactggcacttgcagcacgaaggctgccgcttgcagcatgacgactggcagctgaagcactgcgtcgtgcaggggcacttgcacaggctcgagcagcagccagcgcaggagcatttgaagatgttgaagcagctggagctacgagggcacgagcaggagaagcagtccgcgcagcggcagcactgctcgccgcagcagccgttgcagcttggcttgcagctacagcaattggtagagcacgagccacaacatccgcagcttggacagccacagccgttgatccacagcttggacaggcacagcccttgcagctacagcttggatgggtgcaagtgcagcaattggggctagagcagttgcactgggacttgaagcatgaagggatcttgaagcagttgtacagggatttgaagcatgaaaagatgttgaagcaggtgcagctcggtttgcagcacgatgtatcgcagcatgagctgcagcttggacaggagcacgagcagttcggtttgcagcaagaaggggtcttgcagcaactgcagttaggtttacagcatgagcagcagtctggactacagcatgacccagcgtgtgggtgtgtctggtcttgcaagaattgtacagagggttgtcgatgcccacggtgtcgtaacccatgctgtctcagtggttgcttatgttgatctagatccttttttggttgtcgtttttcttgtattttttagttgttag